(19) United States
(12) Patent Application Publication
(10) Pub. No.: US 2024/0170129 A1
(43) Pub. Date: May 23, 2024

(54) SYSTEM AND METHOD FOR GENERATING A NEONATAL DISORDER NOURISHMENT PROGRAM

(71) Applicant: KPN INNOVATIONS, LLC., LAKEWOOD, CO (US)

(72) Inventor: Kenneth Neumann, LAKEWOOD, CO (US)

(73) Assignee: KPN INNOVATIONS, LLC., LAKEWOOD, CO (US)

(21) Appl. No.: 18/426,154

(22) Filed: Jan. 29, 2024

Related U.S. Application Data
(63) Continuation-in-part of application No. 17/187,970, filed on Mar. 1, 2021, now Pat. No. 11,935,642.

(51) Int. Cl. G16H 20/60; A61B 5/00; A61B 5/145; G16H 50/20

(52) U.S. Cl. CPC G16H 20/60; A61B 5/14542; A61B 5/4812; G16H 50/20; A61B 2503/045

(57) ABSTRACT

A system and method for generating a neonatal disorder nourishment program comprising a computing device, the computing device configured to obtain a neonatal indicator element, identify a neonatal bundle as a function of the neonatal indicator element, produce a neonatal profile as a function of the neonatal bundle, wherein producing further comprises obtaining a neonatal functional goal as a function of the neonatal bundle, receiving a neonatal recommendation as a function of a neonatal database, and producing the neonatal profile as a function of the neonatal functional goal and neonatal recommendation using a neonatal machine-learning model, determine an aliment as a function of the neonatal profile, and generate a nourishment program as a function of the aliment.

### CROSS-REFERENCE TO RELATED APPLICATIONS

This application is a continuation-in-part of Non-provisional application Ser. No. 17/187,970 filed on Mar. 1, 2021, and entitled “SYSTEM AND METHOD FOR GENERATING A NEONATAL DISORDER NOURISHMENT PROGRAM,” the entirety of which is incorporated herein by reference.

### FIELD OF THE INVENTION

The present invention generally relates to the field of artificial intelligence. In particular, the present invention is directed to a system and method for generating a neonatal disorder nourishment program.

### BACKGROUND

Current edible suggestion systems do not account for the status of a newborn. This leads to the inefficiency of a poor nutrition plan for the newborn. This is further complicated by a lack of uniformity of nutritional plans, which results in poor developmental growth.

### SUMMARY OF THE DISCLOSURE

In an aspect, a system for generating a neonatal disorder nourishment program is discussed. The system includes a computing device, wherein the computing device is configured to receive a plurality of infant measurements, obtain a plurality of neonatal indicator elements by determining a plurality of patterns based on the infant measurements, identify a neonatal bundle as a function of the plurality of neonatal indicator elements, update a neonatal profile of an infant as a function of the neonatal bundle, wherein updating the neonatal profile further includes receiving neonatal training data correlating a plurality of neonatal functional goals and a plurality of neonatal recommendations to the neonatal bundle and the neonatal profiles, training a neonatal machine learning model using the neonatal training data, inputting the plurality of neonatal functional goals and the plurality of neonatal recommendations to the trained neonatal machine learning model, and outputting the updated neonatal profile from the trained neonatal machine learning model, determine an aliment as a function of the updated neonatal profile, and generate a nourishment program as a function of the aliment.

In another aspect, a method for generating a neonatal disorder nourishment program is discussed. The method includes using a computing device to receive a plurality of infant measurements, obtain a plurality of neonatal indicator elements by determining a plurality of patterns based on the infant measurements, identify a neonatal bundle as a function of the plurality of neonatal indicator elements, update a neonatal profile of an infant as a function of the neonatal bundle, wherein updating the neonatal profile further includes receiving neonatal training data correlating a plurality of neonatal functional goals and a plurality of neonatal recommendations to the neonatal bundle and the neonatal profiles, training a neonatal machine learning model using the neonatal training data, inputting the plurality of neonatal functional goals and the plurality of neonatal recommendations to the trained neonatal machine learning model, and outputting the updated neonatal profile from the trained neonatal machine learning model, determine an aliment as a function of the updated neonatal profile, and generate a nourishment program as a function of the aliment.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram illustrating an exemplary embodiment of a system for generating a neonatal disorder nourishment program;

FIG. 2 is a block diagram of an exemplary embodiment of a neonatal phase according to an embodiment of the invention;

FIG. 3 is a block diagram of an exemplary embodiment of an aliment directory according to an embodiment of the invention;

FIG. 4 is a block diagram of an exemplary embodiment of nourishment delivery component according to an embodiment of the invention;

FIG. 5 is a block diagram of an exemplary embodiment of a machine-learning module;

FIG. 6 is a process flow diagram illustrating an exemplary embodiment of a method of generating a neonatal disorder nourishment program; and

FIG. 7 is a block diagram of a computing system that can be used to implement any one or more of the methodologies disclosed herein and any one or more portions thereof.

The drawings are not necessarily to scale and may be illustrated by phantom lines, diagrammatic representations, and fragmentary views. In certain instances, details that are not necessary for an understanding of the embodiments or that render other details difficult to perceive may have been omitted.

### DETAILED DESCRIPTION

At a high level, aspects of the present disclosure are directed to systems and methods for generating a nourishment program for neonatal disorders. In an embodiment, this disclosure may obtain one or more neonatal indicator elements that relate to an infant. Aspects of the present disclosure can be used to produce a neonatal profile for an infant that may include identifying a neonatal disorder. Aspects of the present disclosure can also be used to determine an aliment for the neonatal disorder. Aspects of the present disclosure allow for generating a nourishment program. Exemplary embodiments illustrating aspects of the present disclosure are described below in the context of several specific examples.

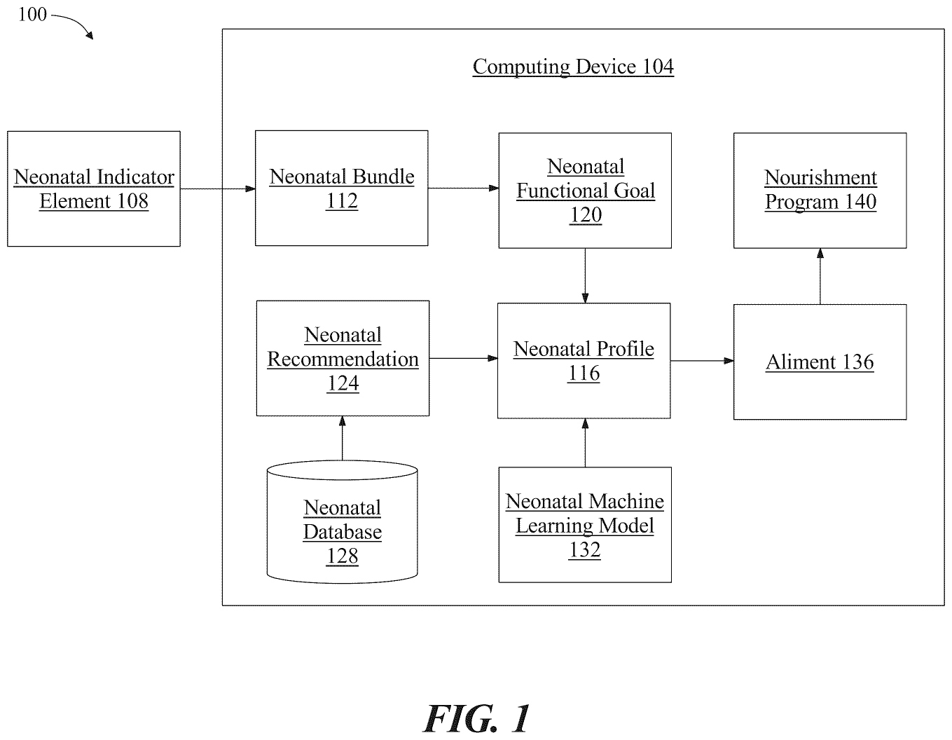

Referring now to FIG. 1, an exemplary embodiment of a system 100 for generating a neonatal disorder nourishment program is illustrated. System includes a computing device 104. computing device 104 may include any computing device as described in this disclosure, including without limitation a microcontroller, microprocessor, digital signal processor (DSP) and/or system on a chip (SoC) as described in this disclosure. Computing device 104 may include, be included in, and/or communicate with a mobile device such as a mobile telephone or smartphone. computing device 104 may include a single computing device operating independently, or may include two or more computing device operating in concert, in parallel, sequentially or the like; two or more computing devices may be included together in a single computing device or in two or more computing devices. computing device 104 may interface or communicate with one or more additional devices as described below in further detail via a network interface device. Network interface devices may be utilized for connecting computing devices 104 to one or more of a variety of networks, and one or more devices. Examples of a network interface device include but are not limited to, a network interface card (e.g., a mobile network interface card, a LAN card), a modem, and any combination thereof. Examples of a network include but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Computing device 104 may include but is not limited to, for example, a computing device or cluster of computing devices in a first location and a second computing device or cluster of computing devices in a second location. computing device 104 may include one or more computing devices dedicated to data storage, security, distribution of traffic for load balancing, and the like. computing device 104 may distribute one or more computing tasks as described below across a plurality of computing devices of computing device, which may operate in parallel, in series, redundantly, or in any other manner used for distribution of tasks or memory between computing devices. computing device 104 may be implemented using a “shared nothing” architecture in which data is cached at the worker, in an embodiment, this may enable scalability of system 100 and/or computing device.

With continued reference to FIG. 1, computing device 104 may be designed and/or configured to perform any method, method step, or sequence of method steps in any embodiment described in this disclosure, in any order and with any degree of repetition. For instance, computing device 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. computing device 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Still referring to FIG. 1, computing device 104 obtains a neonatal indicator clement 108. As used in this disclosure a “neonatal indicator element” is an element of data that denotes a health status of an infant, wherein a health status is a measurement of the relative level of health of an infant. Neonatal indicator element 108 may include a biological sample. As used in this disclosure a “biological sample” is one or more biological specimens collected from an infant. Biological samples may include, without limitation, exhalate, blood, sputum, urine, saliva, feces, semen, and other bodily fluids, as well as tissue. Neonatal indicator clement 108 may include a biological sampling device. Neonatal indicator element 108 may include one or more biomarkers. As used in this disclosure a “biomarker” is a molecule and/or chemical that identifies the health status of an infant. As a non-limiting example, biomarkers may include, bilirubin, poptosis regulator BCL2, breast cancer type 1 susceptibility protein (BRCA1), Fanconi anemia complementation group C (FANCC), vascular endothelial growth factor A (VEGFA), anti-PCNA, anti-SmD, anti-Ro/SSA60, anti-Ro/SSA52, anti-La/SSB, anti-RNPC, S100B, ubiquitin carboxy-terminal hydrolase-L1 [UCH-L1], total Tau, neuron specific enolase, [IL]-1β, IL-6, IL-8, IL-10, IL-12P70, IL-13, interferon-gamma [IFN-γ], tumor necrosis factor alpha [TNF-α], brain-derived neurotrophic factor [BDNF], monocyte chemoattractant protein-1, and the like thereof. As a further non-limiting example, neonatal indicator element 108 may include datum from one or more devices that collect, store, and/or calculate one or more lights, voltages, currents, sounds, chemicals, pressures, and the like thereof that may be capable of monitoring an infant's health status. As a non-limiting example, neonatal indicator element 108 may be obtained as a function of a baby monitor. As a further non-limiting example, neonatal indicator element 108 may be obtained as a function of a baby monitor with a sensing capability. As used in this disclosure a “sensing capability” is one or more capabilities that monitor the health status of an infant. For example, and without limitation sensing capability may include one or more sleep monitoring capabilities, breathing monitoring capabilities, growth tracking capabilities, and the like thereof. Neonatal indicator element 108 may be received as a function of an infant organ system. As used in this disclosure an “infant organ system” is a group of organs and/or tissues that work together as a biological system. For example, and without limitation, an infant organ system may include one or more respiratory systems, digestive systems, excretory systems, circulatory systems, urinary systems, integumentary systems, skeletal systems, muscular systems, endocrine systems, lymphatic systems, nervous systems, reproductive systems, and the like thereof.

Still referring to FIG. 1, a baby monitor may include sensors configured to track a baby's heart rate and oxygen saturation levels. The baby monitor may be equipped with a heart rate sensor that detects the baby's heartbeat. The heart rate sensor may be placed on the baby's skin, such as on the chest or foot, wherein the sensor measures the electrical signals generated by the heart. A heart rate sensor may include a smart sock. A smart sock may include pulse oximetry technology to monitor the baby's heart rate continuously inside a sock housing. The smart sock may measure the subtle changes in blood flow, providing accurate and real-time heart rate information. A heart rate sensor may also include an optical heart rate sensor, electrocardiogram (ECG or EKG) sensor, chest strap heart rate monitor, and the like. The baby monitor may include an oxygen saturation monitor or pulse oximeter. The oxygen saturation monitor may be placed on the baby's skin, such as on a foot or hand, wherein the monitor measures the percentage of oxygen in the baby's blood. An oxygen saturation monitor may include a transmission pulse oximeter, reflectance pulse oximeters, wrist-worn pulse oximeters, and the like. In some embodiments, the baby monitor may include a camera and sleep tracking functionality, wherein the monitor is configured to provide visual and quantitative insights into a baby's sleep patterns. The monitor may be equipped with a high-resolution camera that provides a live video feed of the baby's sleeping area. In addition to the camera, the monitor may include sensors for sleep tracking. These sensors may detect the baby's movements and sleep patterns throughout the night. In some embodiments, a baby monitor may include gas sensors configured to detect specific gases associated with burping or digestion processes, such as flatulence, of a baby. These sensors may detect gases like carbon dioxide (CO2) or other components of expelled air. The monitor be designed as a wearable device attached to the baby's clothing or as a sensor placed near the baby during sleep or feeding times.

Still referring to FIG. 1, all data received from a baby monitor or sensors may be categorized as infant sensor measurements. In some embodiments, neonatal indicator clement 108 may include infant measurements. An “infant measurement,” for the purposes of this disclosure, is a quantitative value relating to the health of an infant. Infant measurements may include any suitable infant measurements as disclosed above. Infant measurements may include user input values. As a non-limiting example, infant measurements may include a number of diaper changes, a number of feedings, a number of naps, a number of burping, and the like. In some embodiments, infant measurements may infant sensor measurements as disclosed in this disclosure. An “infant sensor measurement,” for the purposes of this disclosure is an infant measurement that is made using a sensor.

Still referring to FIG. 1, baby monitor and or computing device 104 may be programmed with pattern recognition algorithms to identify patterns in data detected. For example, a gas sensor may be used to detect burping patterns of a baby. A gas sensor may include a temperature and humidity monitor, breathing monitor, infrared gas sensor, electrochemical gas sensors, metal oxide semiconductor sensors, and the like. Pattern recognition algorithms may include machine learning algorithms, such as Support Vector Machines (SVM), Random Forests, or Neural Networks, to classify, for example, gas patterns into different categories (e.g., normal burping, irregular gas patterns). In an example, a pattern machine learning model may receive an input from the baby monitor and output a pattern, wherein the training data correlates the inputs to a plurality of patterns. A pattern recognition algorithm may iteratively adjust its internal parameters during the training phase to minimize the difference between its predictions and the actual labeled classes in the training dataset. In some embodiments, the algorithm may have adjustable parameters that users can fine-tune based on the specific characteristics of their baby's gas-related activities.

Still referring to FIG. 1, neonatal indicator element 108 may include indicators a of baby's health in relation to heart rate, gas, oxygen saturation levels, and sleep patterns wherein computing device 104 may generate nourishment recommendation, aliments, and the like to improve the baby's health as described further below.

Still referring to FIG. 1, computing device 104 may obtain neonatal indicator element 108 by receiving an input from a user. As used in this disclosure “input” is an element of datum that is obtained as a function of a/an informed advisor, medical advisor, physician, nurse, family member, third-party and the like thereof. As used in this disclosure “informed advisor” is an individual that is skilled in a particular area relating to the study of the infant organ system. As a non-limiting example input may include a nurse entering input that the infant's skin was turning yellow. As a further non-limiting example, input may include a physician entering input that the infant is not vocalizing and/or crying when being stimulated. As a further non-limiting example, inputs may include one or more inputs associated with fussiness, decreased level of consciousness, abnormal movements, feeding difficulty, changes in body temperature, rapid changes in head size, changes in muscle tone, and the like thereof. An input may include photos of soiled diapers, vomit, and other forms of excrement. Input may include one or more inputs from a function of a medical assessment, wherein a “medical assessment” is an evaluation and/or estimation of the health status of an infant. As a non-limiting example medical assessment may include an encephalographic measurement, magnetic resonance image, computed tomographic image, electroencephalogram measurement, electromyographic measurement, Apgar test, Guthrie test, tandem mass spectrometric measurement, nuclear medicine renal scan, gene test, and the like thereof.

Still referring to FIG. 1, in some embodiments, computing device 104 may include receiving a prenatal input. A “prenatal input,” as used herein is information related to the health of a mother during pregnancy. A prenatal input may include information related to the mother's health, the stage of pregnancy, the specific requirements for the developing fetus, and the like. The prenatal input may be used to determine nutritional needs of a baby prenatal. The prenatal input may include the mother's nutritional status and dietary habits, dietary guidance provided by a health care provider, lifestyle and behavioral factors, such as physical activity, and the like. The prenatal input may be received from an informed advisor as described above. In some embodiments, a prenatal input may be used in the addition or alternative to neonatal indicator element 108 to determine nutritional needs, recommendations, aliments, and the like as described in this disclosure.

Still referring to FIG. 1, computing device 104 identifies a neonatal bundle 112 as a function of neonatal indicator clement 108. As used in this disclosure a “neonatal bundle” is a group of neonatal indicator elements that relate to one or more functions of the organ systems of an infant. As a non-limiting example, neonatal bundle 112 may include a medical bundle. As used in this disclosure a “medical bundle” is a bundle of neonatal indicator elements that relate to a medical condition. For example, and without limitation medical bundle may include blood conditions, complex birth defects, genetic conditions, central nervous system conditions, gastrointestinal conditions, heart conditions, metabolic conditions, renal conditions, respiratory conditions, and the like thereof. As a non-limiting example, neonatal bundle 112 may include a surgical bundle. As used in this disclosure a “surgical bundle” is a bundle of neonatal indicator elements that relate to a condition that may require surgery. For example, and without limitation surgical bundle may include airway surgical conditions, gastrointestinal surgical conditions, neurosurgical conditions, orthopedic surgical conditions, urological surgical conditions, and the like thereof.

Still referring to FIG. 1, computing device 104 produces a neonatal profile 116 as a function of neonatal bundle 112. In some embodiments, “producing a neonatal profile” may include updating a neonatal profile. For example, this may include using a pre-existing neonatal profile. In some embodiments, computing device 104 may receive a preexisting neonatal profile from a user or a database, such as, as a non-limiting example, neonatal database 128. As used in this disclosure a “neonatal profile” is a profile of the health status of an infant, wherein a health status is a relative level of wellness and illness of the infant as described above in detail. As a non-limiting example neonatal profile 116 may include a profile comprising a birth weight, Apgar score, neonatal length, and the like thereof. As a further non-limiting example, neonatal profile 116 may include a profile comprising birth weight, congenital anomalies, perinatal problems, and the like thereof. In an embodiment, and without limitation, neonatal profile may include an aliment intolerance. As used in this disclosure an “aliment intolerance” is a difficulty digesting a particular aliment. For example, and without limitation, aliment intolerance may include one or more sensitivities to lactose, gluten, caffeine, salicylates, amines, FODMAPs, sulfites, fructose, aspartame, eggs, MSG, food colorings, yeast, sugar alcohols, and the like thereof. As a further non-limiting example, an aliment intolerance may be a result of an infant lacking an enzyme needed to digest an aliment and/or absorb nutrients from an aliment. Computing device 104 produces neonatal profile 116 by obtaining a neonatal functional goal 120 as a function of neonatal bundle 112. As used in this disclosure “neonatal functional goal” is an intended function of a neonatal bundle. For example, and without limitation neonatal functional goal 120 may include an intended goal of improving function of a circulatory system blood pressure to be enhanced and/or raised. As a further non-limiting example, neonatal functional goal 120 may include an intended goal of improving function of a respiratory system to include enhancing and/or raising a breathing rate of an infant. As a further non-limiting example, neonatal functional goal 120 may include an intended goal of improving function of a urinary system to enhance the filtration rate of an infant.

Still referring to FIG. 1, computing device 104 may produce neonatal profile 116 by receiving a neonatal recommendation 124. As used in this disclosure a “neonatal recommendation” is a recommendation and/or guideline associated with a health status of an infant. As a non-limiting example, neonatal recommendation 124 may include one or more recommendations and/or guidelines as a function of improving the health status of an infant. As a further non-limiting example, neonatal recommendation 124 may include one or more recommendations and/or guidelines as a function of maintaining a present health status of an infant. As a non-limiting example, neonatal recommendation 124 may include a recommendation that an Apgar Test be greater than 7. As a further non-limiting example, neonatal recommendation 124 may include a recommendation that bilirubin concentrations be less than 5.2 mg/dL. Neonatal recommendation 124 is received as a function of a neonatal database 128. As used in this disclosure a “neonatal database” is a database of recommendations associated with the health status of an infant. In an embodiment, and without limitation, neonatal database 128 may include information from a peer review, an advisor association, a medical website, and the like thereof. As a non-limiting example, integumentary database 128 may include information from one or more articles and/or publications from the Journal of Neonatology, Journal of Neonatal Nursing, Advances in Neonatal care, and the like thereof. As a non-limiting example, integumentary database 128 may include information from one or more advisor associations such as, but not limited to, the National Association of Neonatal Nurses, The Organization for Neonatal Nurses, Academy of Neonatal Nursing, Association of Women's Health, Obstetric, and Neonatal Nurses. As a further non-limiting example, neonatal recommendation 124 may include one or more recommendations from the World Health Organization. As a non-limiting example, World Health Organization may recommend that serum ferritin concentrations should exceed 150 ug/dL. As a further non-limiting example, World Health Organization may recommend that IL-6 concentrations should be less than 18.3 pg/mL. As a further non-limiting example, neonatal recommendation 124 may include a glomerular filtration rate of 20 mL/min/1.73 m². As a further non-limiting example, neonatal recommendation 124 may include a systolic pressure of 64 mmHg and a diastolic pressure of 41 mmHg. As a further non-limiting example, neonatal recommendation 124 may include of 40 breaths per minute.

Still referring to FIG. 1, computing device 104 produces/updates neonatal profile 116 as a function of neonatal functional goal 120 and neonatal recommendation 124 using a neonatal machine-learning model 132. As used in this disclosure “neonatal machine-learning model” is a machine-learning model to produce a neonatal profile output given neonatal functional goals and neonatal recommendations as inputs; this is in contrast to a non-machine learning software program where the commands to be executed are determined in advance by a user and written in a programming language. Neonatal machine-learning model 132 may include one or more neonatal machine-learning processes such as supervised, unsupervised, or reinforcement machine-learning processes that computing device 104 and/or a remote device may or may not use in the determination of neonatal profile 116. As used in this disclosure “remote device” is an external device to computing device 104. An neonatal machine-learning process may include, without limitation machine learning processes such as simple linear regression, multiple linear regression, polynomial regression, support vector regression, ridge regression, lasso regression, elasticnet regression, decision tree regression, random forest regression, logistic regression, logistic classification, K-nearest neighbors, support vector machines, kernel support vector machines, naïve bayes, decision tree classification, random forest classification, K-means clustering, hierarchical clustering, dimensionality reduction, principal component analysis, linear discriminant analysis, kernel principal component analysis, Q-learning, State Action Reward State Action (SARSA), Deep-Q network, Markov decision processes, Deep Deterministic Policy Gradient (DDPG), or the like thereof.

Still referring to FIG. 1, computing device 104 may train neonatal machine-learning process as a function of a neonatal training set. As used in this disclosure “neonatal training set” is a training set that correlates a neonatal functional goal and/or neonatal recommendation to a neonatal profile. In some embodiments, such as when neonatal profile 116 is being updated, neonatal training set may include neonatal profiles correlated and neonatal functional goals and/or neonatal recommendations correlated to updated neonatal profiles. In some embodiments, when updating neonatal profile 116, neonatal machine-learning model may be configured to receive neonatal profile 116 as input and to output an updated neonatal profile. In some embodiments, neonatal machine-learning model may be configured also receive neonatal functional goals and/or neonatal recommendations as input along with the neonatal profile. For example, and without limitation, a neonatal functional goal of reducing bilirubin concentration and a neonatal recommendation of a bilirubin concentration to be less than 18.3 pg/mL may relate to a neonatal profile of obstructed bile duct. Neonatal training set may be received as a function of user-entered valuations of neonatal functional goals, neonatal recommendations, and/or neonatal profiles. Computing device 104 may receive neonatal training set by receiving correlations of neonatal functional goals, and/or neonatal recommendations that were previously received and/or determined during a previous iteration of determining neonatal profiles. Neonatal training set may be received by one or more remote devices that at least correlate a neonatal functional goal and/or neonatal recommendation to a neonatal profile, wherein a remote device is an external device to computing device 104, as described above. Neonatal training set may be received in the form of one or more user-entered correlations of a neonatal functional goal and/or neonatal recommendation to a neonatal profile. A user may include an informed advisor, wherein an informed advisor may include, without limitation, neonatologists, pediatricians, family physicians, and the like thereof.

Still referring to FIG. 1, computing device 104 may receive neonatal machine-learning model from a remote device that utilizes one or more neonatal machine learning processes, wherein a remote device is described above in detail. For example, and without limitation, a remote device may include a computing device, external device, processor, and the like thereof. Remote device may perform the neonatal machine-learning process using the neonatal training set to generate neonatal profile 116 and transmit the output to computing device 104. Remote device may transmit a signal, bit, datum, or parameter to computing device 104 that at least relates to neonatal profile 116. Additionally or alternatively, the remote device may provide an updated machine-learning model. For example, and without limitation, an updated machine-learning model may be comprised of a firmware update, a software update, a neonatal machine-learning process correction, and the like thereof. As a non-limiting example a software update may incorporate a new neonatal functional goal that relates to a modified neonatal recommendation. Additionally or alternatively, the updated machine learning model may be transmitted to the remote device, wherein the remote device may replace the neonatal machine-learning model with the updated machine-learning model and determine the neonatal profile as a function of the neonatal functional goal using the updated machine-learning model. The updated machine-learning model may be transmitted by the remote device and received by computing device 104 as a software update, firmware update, or corrected neonatal machine-learning model. For example, and without limitation neonatal machine-learning model may utilize a random forest machine-learning process, wherein the updated machine-learning model may incorporate a gradient boosting machine-learning process. Updated machine learning model may additionally or alternatively include any machine-learning model used as an updated machine learning model as described in U.S. Nonprovisional application Ser. No. 17/106,658, filed on Nov. 30, 2020, and entitled “A SYSTEM AND METHOD FOR GENERATING A DYNAMIC WEIGHTED COMBINATION,” the entirety of which is incorporated herein by reference.

In an embodiment and without limitation, neonatal machine-learning model 132 may include a classifier. A “classifier,” as used in this disclosure is a machine-learning model, such as a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. Computing device 104 and/or another device may generate a classifier using a classification algorithm, defined as a processes whereby a computing device 104 derives a classifier from training data. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers.

Still referring to FIG. 1, computing device 104 may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)÷P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Computing device 104 may then calculate a likelihood table by calculating probabilities of different data entries and classification labels. Computing device 104 may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 1, computing device 104 may be configured to generate a classifier using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 1, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least one value. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute l as derived using a Pythagorean norm: l=\sqrt{Σᵢ₌₀ⁿaᵢ²}, where aᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

Still referring to FIG. 1, computing device 104 may identify neonatal profile 116 by identifying a neonatal disorder. As used in this disclosure a “neonatal disorder” is an ailment and/or collection of ailments that impact an infant's organ system. As a non-limiting example, neonatal disorder may include Hyperbilirubinemia, jaundice, thrombocytopenia, hypoxic ischemic encephalopathy, seizures, head/body cooling, hypotonia, muscle disorders, feeding intolerance, GERD, failure to thrive, anemia of prematurity, apnea of prematurity, atrial septal defect, atrioventricular septal defect, benign neonatal hemangiomatosis, brachial plexus injury, bronchopulmonary dysplasia, cerebral palsy, coarctation of the aorta, congenital adrenal hyperplasia, congenital diaphragmatic hernia, congenital heart disease, diffuse neonatal hemangiomatosis, encephalocele, gastroschisis, hemolytic disease of the newborn, lissencephaly, omphalocele, patent ductus arteriosus, perinatal asphyxia, periventricular leukomalacia, persistent pulmonary hypertension of the newborn, persistent truncus arteriosus, pulmonary hypoplasia, retinopathy of prematurity, spina bifida, spinal muscular atrophy, supraventricular tachycardia, tetralogy of Fallot, tracheoesophageal fistula, tricuspid atresia, trisomy 13/18/21, ventricular septal defects, and the like thereof. Neonatal disorder may be determined as a function of one or more disorder machine-learning models. As used in this disclosure, a “disorder machine-learning model” is a machine-learning model to produce a neonatal disorder output given neonatal indicator element 108 and/or neonatal bundle 112 as inputs; this is in contrast to a non-machine learning software program where the commands to be executed are determined in advance by a user and written in a programming language. Disorder machine-learning models may include one or more disorder machine-learning processes such as supervised, unsupervised, or reinforcement machine-learning processes that computing device 104 and/or a remote device may or may not use in the determination of neonatal disorder. A disorder machine-learning process may include, without limitation machine learning processes such as simple linear regression, multiple linear regression, polynomial regression, support vector regression, ridge regression, lasso regression, elasticnet regression, decision tree regression, random forest regression, logistic regression, logistic classification, K-nearest neighbors, support vector machines, kernel support vector machines, naïve bayes, decision tree classification, random forest classification, K-means clustering, hierarchical clustering, dimensionality reduction, principal component analysis, linear discriminant analysis, kernel principal component analysis, Q-learning, State Action Reward State Action (SARSA), Deep-Q network, Markov decision processes, Deep Deterministic Policy Gradient (DDPG), or the like thereof.

Still referring to FIG. 1, computing device 104 may train disorder machine-learning process as a function of a disorder training set. As used in this disclosure, a “disorder training set” is a training set that correlates at least a neonatal enumeration and an infant organ system effect to a neonatal disorder. As used in this disclosure, an “neonatal enumeration” is a measurable value associated with the neonatal indicator clement. As used in this disclosure, a “neonatal system effect” is an impact and/or effect the neonatal bundle has on the neonatal system of an infant. As a non-limiting example, a neonatal enumeration of 23 may be related to a neonatal system effect of blood in bowel movements wherein a neonatal disorder of necrotizing enterocolitis may be determined. The disorder training set may be received as a function of user-entered valuations of neonatal enumerations, neonatal system effects, and/or neonatal disorders. Computing device 104 may receive disorder training set by receiving correlations of neonatal enumerations and/or neonatal system effects that were previously received and/or determined during a previous iteration of determining neonatal disorders. The disorder training set may be received by one or more remote devices that at least correlate a neonatal enumeration and/or neonatal system effect to a neonatal disorder, wherein a remote device is an external device to computing device 104, as described above. The disorder training set may be received in the form of one or more user-entered correlations of a neonatal enumeration and neonatal system effect to a neonatal disorder. Additionally or alternatively, a user may include an informed advisor, wherein an informed advisor may include, without limitation, neonatologists, pediatricians, family physicians, and the like thereof.

Still referring to FIG. 1, computing device 104 may receive disorder machine-learning model from the remote device that utilizes one or more disorder machine learning processes, wherein a remote device is described above in detail. For example, and without limitation, a remote device may include a computing device, external device, processor, and the like thereof. The remote device may perform the disorder machine-learning process using the disorder training set to generate neonatal disorder and transmit the output to computing device 104. The remote device may transmit a signal, bit, datum, or parameter to computing device 104 that at least relates to neonatal disorders. Additionally or alternatively, the remote device may provide an updated machine-learning model. For example, and without limitation, an updated machine-learning model may be comprised of a firmware update, a software update, a disorder machine-learning process correction, and the like thereof. As a non-limiting example a software update may incorporate a new neonatal enumeration that relates to a modified neonatal system effect. Additionally or alternatively, the updated machine learning model may be transmitted to the remote device, wherein the remote device may replace the disorder machine-learning model with the updated machine-learning model and determine the neonatal disorder as a function of the neonatal enumeration using the updated machine-learning model. The updated machine-learning model may be transmitted by the remote device and received by computing device 104 as a software update, firmware update, or corrected disorder machine-learning model. For example, and without limitation disorder machine-learning model may utilize a Naïve bayes machine-learning process, wherein the updated machine-learning model may incorporate decision tree machine-learning process.

Still referring to FIG. 1, determining a neonatal disorder may include an image-based waste machine learning model configured to receive neonatal indicator elements 108 including photos of a baby's excretions and waste and output of a neonatal disorder. Waste machine learning model training data may include data correlating a plurality of waste related photos to a plurality of neonatal disorders. Waste machine learning model may include image processing techniques in order to extract data from photos and classify to a disorder. Images may be preprocessed to enhance features and make them suitable for input into the waste machine learning model. This may include tasks like resizing, normalization, and color adjustments. Image processing may include feature extraction processes such as Histogram of Oriented Gradients (HOG), Scale-Invariant Feature Transform (SIFT), Local Binary Patterns (LBP), color histograms, auto encoders and the like. For example, a feature extraction process may be used to identify elements such as blood, discoloration, foreign objects in stool to classify those elements to a waste related neonatal disorder. A waste related neonatal disorder may include fecal, urine, and excretions disorder classifications such as diarrhea, constipation, and the like.

Still referring to FIG. 1, a waste machine learning model may incorporate a machine-vision system to extract and analyze data. A machine vision system may use images from at least a camera, to make a determination about a scene, space, and/or object. For example, in some cases a machine vision system may be used for world modeling or registration of objects within a space. In some cases, registration may include image processing, such as without limitation object recognition, feature detection, edge/corner detection, and the like. Non-limiting examples of feature detection may include scale invariant feature transform (SIFT), Canny edge detection, Shi Tomasi corner detection, and the like. In some cases, registration may include one or more transformations to orient a camera frame (or an image or video stream) relative a three-dimensional coordinate system; exemplary transformations include without limitation homography transforms and affine transforms. In an embodiment, registration of first frame to a coordinate system may be verified and/or corrected using object identification and/or computer vision, as described above. For instance, and without limitation, an initial registration to two dimensions, represented for instance as registration to the x and y coordinates, may be performed using a two-dimensional projection of points in three dimensions onto a first frame, however. A third dimension of registration, representing depth and/or a z axis, may be detected by comparison of two frames; for instance, where first frame includes a pair of frames captured using a pair of cameras (e.g., stereoscopic camera also referred to in this disclosure as stereo-camera), image recognition and/or edge detection software may be used to detect a pair of stereoscopic views of images of an object; two stereoscopic views may be compared to derive z-axis values of points on object permitting, for instance, derivation of further z-axis points within and/or around the object using interpolation. This may be repeated with multiple objects in field of view, including without limitation environmental features of interest identified by object classifier and/or indicated by an operator. In an embodiment, x and y axes may be chosen to span a plane common to two cameras used for stereoscopic image capturing and/or an xy plane of a first frame; a result, x and y translational components and ϕ may be pre-populated in translational and rotational matrices, for affine transformation of coordinates of object, also as described above. Initial x and y coordinates and/or guesses at transformational matrices may alternatively or additionally be performed between first frame and second frame, as described above. For each point of a plurality of points on object and/or edge and/or edges of object as described above, x and y coordinates of a first stereoscopic frame may be populated, with an initial estimate of z coordinates based, for instance, on assumptions about object, such as an assumption that ground is substantially parallel to an xy plane as selected above. Z coordinates, and/or x, y, and z coordinates, registered using image capturing and/or object identification processes as described above may then be compared to coordinates predicted using initial guess at transformation matrices; an error function may be computed using by comparing the two sets of points, and new x, y, and/or z coordinates, may be iteratively estimated and compared until the error function drops below a threshold level. In some cases, a machine vision system may use a classifier, such as any classifier described throughout this disclosure.

Still referring to FIG. 1, computing device 104 may be configured to determine a waste remedy for a neonatal disorder related to waste. A “waste remedy,” is a protocol to combat a neonatal disorder. A waste remedy may be a protocol that may be performed by an informed advisor to a baby during a diaper change, feeding bathing, and the like. For example, a waste remedy may include giving a baby warm bathes to soothe and relax the baby's tense muscles in relation to a constipation neonatal disorder. A waste remedy may be generated using a remedy machine learning model configured to receive the waste related neonatal disorder related and output the waste remedy. Waste remedy machine learning model training data may include data correlating a waste related neonatal disorder to a plurality of waste remedies. In some embodiments, computing device may record waste related neonatal phases 108, neonatal disorder, and wastes remedies received and/or generated over a period time to determine nutritional recommendations, aliments, and the like as described further below.

Still referring to FIG. 1, computing device 104 may use a waste remedy lookup table to identify a waste remedy. A “lookup table,” as used in this disclosure, is an array of data that maps input values to output values, thereby approximating a mathematical function. Given a set of input values, a lookup operation retrieves the corresponding output values from the table. A lookup table may be populated with waste remedies correlated to a disorders. If the lookup table does not explicitly define the input values, computing device 104 can estimate an output value using interpolation, extrapolation, or rounding, wherein interpolation is a process for estimating values that lie between known data points; extrapolation is a process for estimating values that lie beyond the range of known data points; and a rounding is a process for approximating a value by altering its digits according to a known rule. In some embodiments, computing device may look up neonatal disorder related to waste in the lookup table to find an associated waste remedy.

Still referring to FIG. 1, computing device 104 may be configured to classify a user such as a parental figure of a baby, families, and the like to plurality of users matched with the same or similar neonatal disorder. For example, computing device 104 may train a cohort machine-learning model to receive a neonatal profile 116 and/or a neonatal disorder and output list of users. The list of users may include contact information such as email, phone number, online handle, and the like, The list user may include neonatal profiles 116 and/or neonatal disorders that match the current user of apparatus 100. Training data for the cohort machine-learning model may include data correlating a neonatal profile and a neonatal disorder to a cohort of neonatal profiles and neonatal disorders.

Still referring to FIG. 1, a cohort machine-learning model may include clustering algorithms to group users. A clustering algorithm may be implemented, as a non-limiting example, using a k-means clustering algorithm. A “k-means clustering algorithm” as used in this disclosure, includes cluster analysis that partitions n observations or unclassified cluster data entries into k clusters in which each observation or unclassified cluster data entry belongs to the cluster with the nearest mean. “Cluster analysis” as used in this disclosure, includes grouping a set of observations or data entries in way that observations or data entries in the same group or cluster are more similar to each other than to those in other groups or clusters. Cluster analysis may be performed by various cluster models that include connectivity models such as hierarchical clustering, centroid models such as k-means, distribution models such as multivariate normal distribution, density models such as density-based spatial clustering of applications with nose (DBSCAN) and ordering points to identify the clustering structure (OPTICS), subspace models such as biclustering, group models, graph-based models such as a clique, signed graph models, neural models, and the like. Cluster analysis may include hard clustering whereby each observation or unclassified cluster data entry belongs to a cluster or not. Cluster analysis may include soft clustering or fuzzy clustering whereby each observation or unclassified cluster data entry belongs to each cluster to a certain degree such as for example a likelihood of belonging to a cluster; for instance, and without limitation, a fuzzy clustering algorithm may be used to identify clustering of elements of a first type or category with elements of a second type or category, and vice versa. Cluster analysis may include strict partitioning clustering whereby each observation or unclassified cluster data entry belongs to exactly one cluster. Cluster analysis may include strict partitioning clustering with outliers whereby observations or unclassified cluster data entries may belong to no cluster and may be considered outliers. Cluster analysis may include overlapping clustering whereby observations or unclassified cluster data entries may belong to more than one cluster. Cluster analysis may include hierarchical clustering whereby observations or unclassified cluster data entries that belong to a child cluster also belong to a parent cluster.

With continued reference to FIG. 1, computing device 104 may generate a k-means clustering algorithm receiving unclassified data and outputs a definite number of classified data entry clusters wherein the data entry clusters each contain cluster data entries. K-means algorithm may select a specific number of groups or clusters to output, identified by a variable “k.” Generating a k-means clustering algorithm includes assigning inputs containing unclassified data to a “k-group” or “k-cluster” based on feature similarity. Centroids of k-groups or k-clusters may be utilized to generate classified data entry cluster. K-means clustering algorithm may select and/or be provided “k” variable by calculating k-means clustering algorithm for a range of k values and comparing results. K-means clustering algorithm may compare results across different values of k as the mean distance between cluster data entries and cluster centroid. K-means clustering algorithm may calculate mean distance to a centroid as a function of k value, and the location of where the rate of decrease starts to sharply shift, this may be utilized to select a k value. Centroids of k-groups or k-cluster include a collection of feature values which are utilized to classify data entry clusters containing cluster data entries. K-means clustering algorithm may act to identify clusters of closely related data, which may be provided with user cohort labels; this may, for instance, generate an initial set of user cohort labels from an initial set of data, and may also, upon subsequent iterations, identify new clusters to be provided new labels, to which additional data may be classified, or to which previously used data may be reclassified.

With continued reference to FIG. 1, generating a k-means clustering algorithm may include generating initial estimates for k centroids which may be randomly generated or randomly selected from unclassified data input. K centroids may be utilized to define one or more clusters. K-means clustering algorithm may assign unclassified data to one or more k-centroids based on the squared Euclidean distance by first performing a data assigned step of unclassified data. K-means clustering algorithm may assign unclassified data to its nearest centroid based on the collection of centroids ci of centroids in set C. Unclassified data may be assigned to a cluster based on [symbol] dist(ci, x)², where argmin includes argument of the minimum, ci includes a collection of centroids in a set C, and dist includes standard Euclidean distance. K-means clustering module may then recompute centroids by taking means of all cluster data entries assigned to a centroid's cluster. This may be calculated based on ci=1/|Si|Σxi [symbol] Si^{xi}. K-means clustering algorithm may continue to repeat these calculations until a stopping criterion has been satisfied such as when cluster data entries do not change clusters, the sum of the distances have been minimized, and/or some maximum number of iterations has been reached.

Still referring to FIG. 1, k-means clustering algorithm may be configured to calculate a degree of similarity index value. A “degree of similarity index value” as used in this disclosure, includes a distance measurement indicating a measurement between each data entry cluster generated by k-means clustering algorithm and a selected element. Degree of similarity index value may indicate how close a particular combination of elements is to being classified by k-means algorithm to a particular cluster. K-means clustering algorithm may evaluate the distances of the combination of elements to the k-number of clusters output by k-means clustering algorithm. Short distances between an element of data and a cluster may indicate a higher degree of similarity between the element of data and a particular cluster. Longer distances between an element and a cluster may indicate a lower degree of similarity between an element to be compared and/or clustered and a particular cluster.

With continued reference to FIG. 1, k-means clustering algorithm selects a classified data entry cluster as a function of the degree of similarity index value. In an embodiment, k-means clustering algorithm may select a classified data entry cluster with the smallest degree of similarity index value indicating a high degree of similarity between an element and the data entry cluster. Alternatively or additionally k-means clustering algorithm may select a plurality of clusters having low degree of similarity index values to elements to be compared and/or clustered thereto, indicative of greater degrees of similarity. Degree of similarity index values may be compared to a threshold number indicating a minimal degree of relatedness suitable for inclusion of a set of element data in a cluster, where degree of similarity indices a-n falling under the threshold number may be included as indicative of high degrees of relatedness. The above-described illustration of feature learning using k-means clustering is included for illustrative purposes only, and should not be construed as limiting potential implementation of feature learning algorithms; persons skilled in the art, upon reviewing the entirety of this disclosure, will be aware of various additional or alternative feature learning approaches that may be used consistently with this disclosure.

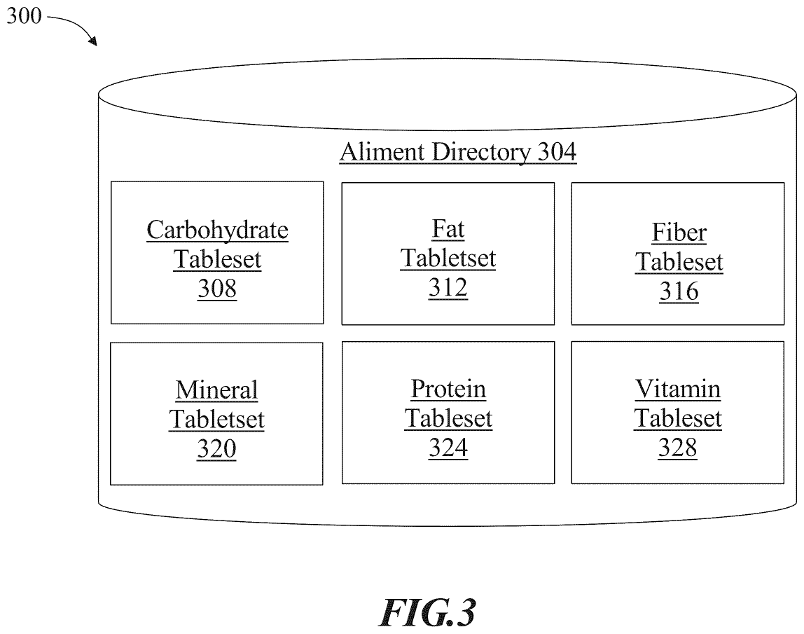

Still referring to FIG. 1, computing device 104 determines an aliment 136 as a function of neonatal profile 116. As used in this disclosure an “aliment” is a source of nourishment that may be provided to an infant such that the infant may absorb the nutrients from the source. For example, and without limitation, an aliment may include formula, breastmilk, dissolved foods, nutrient mixtures, and the like thereof. Computing device 104 may determine aliment 136 as a function of receiving a nourishment composition. As used in this disclosure a “nourishment composition” is a list and/or compilation of all of the nutrients contained in an aliment. As a non-limiting example nourishment composition may include one or more quantities and/or amounts of total fat, including saturated fat and/or trans-fat, cholesterol, sodium, total carbohydrates, including dietary fiber and/or total sugars, protein, vitamin A, vitamin C, thiamin, riboflavin, niacin, pantothenic acid, vitamin b6, folate, biotin, vitamin B12, vitamin D, vitamin E, vitamin K, calcium, iron, phosphorous, iodine, magnesium, zinc, selenium, copper, manganese, chromium, molybdenum, chloride, and the like thereof. Nourishment composition may be obtained as a function of an aliment directory, wherein an “aliment directory” is a database of aliments that may be identified as a function of one or more neonatal indicator elements, as described in detail below, in reference to FIG. 3.

Still referring to FIG. 1, computing device 104 may produce a nourishment demand as a function of neonatal profile 116. As used in this disclosure a “nourishment demand” is requirement and/or necessary amount of nutrients required for an infant to receive. As a non-limiting example, nourishment demand may include an infant requirement of 9 kcal of lipids to be consumed per day. Nourishment demand may be determined as a function of receiving a nourishment goal. As used in this disclosure a “nourishment goal” is a recommended amount of nutrients that an infant should consume. Nourishment goal may be identified by one or more organizations that relate to, represent, and/or study neonatal conditions, such as the National Association of Neonatal Nurses, The Organization for Neonatal Nurses, Academy of Neonatal Nursing, Association of Women's Health, Obstetric, and Neonatal Nurses, and the like thereof.

Still referring to FIG. 1, computing device 104 may determine aliment 136 as a function of nourishment composition, nourishment demand, and an aliment machine-learning model. As used in this disclosure an “aliment machine-learning model” is a machine-learning model to produce an aliment output given nourishment compositions and nourishment demands as inputs; this is in contrast to a non-machine learning software program where the commands to be executed are determined in advance by a user and written in a programming language. Aliment machine-learning model may include one or more aliment machine-learning processes such as supervised, unsupervised, or reinforcement machine-learning processes that computing device 104 and/or a remote device may or may not use in the determination of aliment 136, wherein a remote device is an external device to computing device 104 as described above in detail. An aliment machine-learning process may include, without limitation machine learning processes such as simple linear regression, multiple linear regression, polynomial regression, support vector regression, ridge regression, lasso regression, elasticnet regression, decision tree regression, random forest regression, logistic regression, logistic classification, K-nearest neighbors, support vector machines, kernel support vector machines, naïve bayes, decision tree classification, random forest classification, K-means clustering, hierarchical clustering, dimensionality reduction, principal component analysis, linear discriminant analysis, kernel principal component analysis, Q-learning, State Action Reward State Action (SARSA), Deep-Q network, Markov decision processes, Deep Deterministic Policy Gradient (DDPG), or the like thereof.

Still referring to FIG. 1, computing device 104 may train aliment machine-learning process as a function of an aliment training set. As used in this disclosure an “aliment training set” is a training set that correlates at least nourishment composition and nourishment demand to an aliment. For example, and without limitation, nourishment composition of 8 g of protein and a nourishment demand of 4 g/kg of protein as a function of neonatal anemia may relate to an aliment of fortified milk. The aliment training set may be received as a function of user-entered valuations of nourishment compositions, nourishment demands, and/or aliments. Computing device 104 may receive aliment training set by receiving correlations of nourishment compositions and/or nourishment demands that were previously received and/or determined during a previous iteration of determining aliments. The aliment training set may be received by one or more remote devices that at least correlate a nourishment composition and nourishment demand to an aliment, wherein a remote device is an external device to computing device 104, as described above. Aliment training set may be received in the form of one or more user-entered correlations of a nourishment composition and/or nourishment demand to an aliment. Additionally, or alternatively, a user may include an informed advisor, wherein an informed advisor may include, without limitation, dermatologists, functional medicine practitioners, chemical pathologists, family physicians, family physicians, and the like thereof. Additionally, or alternatively, aliment machine-learning model 148 may identify aliment 136 as a function of one or more classifiers, wherein classifiers are described above in detail.

Still referring to FIG. 1, computing device 104 may receive aliment machine-learning model 148 from a remote device that utilizes one or more aliment machine learning processes, wherein remote device is described above in detail. For example, and without limitation, remote device may include a computing device, external device, processor, and the like thereof. Remote device may perform the aliment machine-learning process using the aliment training set to generate aliment 136 and transmit the output to computing device 104. Remote device may transmit a signal, bit, datum, or parameter to computing device 104 that at least relates to aliment 136. Additionally or alternatively, the remote device may provide an updated machine-learning model. For example, and without limitation, an updated machine-learning model may be comprised of a firmware update, a software update, an aliment machine-learning process correction, and the like thereof. As a non-limiting example a software update may incorporate a new nourishment composition that relates to a modified nourishment demand. Additionally or alternatively, the updated machine learning model may be transmitted to the remote device, wherein the remote device may replace the aliment machine-learning model with the updated machine-learning model and determine the aliment as a function of the nourishment demand using the updated machine-learning model. The updated machine-learning model may be transmitted by the remote device and received by computing device 104 as a software update, firmware update, or corrected aliment machine-learning model. For example, and without limitation an aliment machine-learning model may utilize a neural net machine-learning process, wherein the updated machine-learning model may incorporate polynomial regression machine-learning process. Updated machine learning model may additionally or alternatively include any machine-learning model used as an updated machine learning model as described in U.S. Nonprovisional application Ser. No. 17/106,658, filed on Nov. 30, 2020, and entitled “A SYSTEM AND METHOD FOR GENERATING A DYNAMIC WEIGHTED COMBINATION,” the entirety of which is incorporated herein by reference.

With continued reference to FIG. 1, computing device 104 may be configured to determine a neonatal phase 108. A “neonatal phase,” as used in this disclosure, is any data describing an infant life stage. An infant life stage may be marked by one or more characteristics of a developing infant. An infant life stage may include a particular time period, such as a day, week, month, year, and the like thereof. During an infant life stage, an infant may modify and/or develop one or more biomedical, behavioral, and/or social developments, such as motor functions, sounds, cognitive functions, and the like thereof. Computing device 104 may calculate neonatal phase by receiving an age datum. An “age datum,” as used in this disclosure, is any data that is utilized to calculate neonatal phase. Age datum may describe an infant's life stage development which may be calculated by an informed advisor, wherein an informed advisor is described in detail above. For example, a physician may calculate an infant's life stage development by analyzing a blood analysis from an infant. Age datum may describe an infant's conception date which may indicate a possible range of days during which a user's baby was conceived whether using artificial or natural methods to assist in determining a nutrient requirement as a function of the conception date. For example, a date of conception may reflect a range of days during which sexual intercourse may have led to conception, wherein the date of conception may alter the nutrient demand of the infant. Age datum may describe one or more measurements obtained from an ultrasound such as a fundal height measurement or a size measurement.

With continued reference to FIG. 1, computing device 104 may be configured to classify an age datum to a neonatal progression level. As used in this disclosure a “neonatal progression level” is a level at which the infant should be at in relation to the age datum. For example, and without limitation, age datum may be received that identifies an infant at age 2 months old, wherein the neonatal progression level identifies that the development of the infant is only at 1 month. As a further non-limiting example, age datum may be received that identifies an infant at age 34 weeks old, wherein the neonatal progression level identifies that the development of the infant is 42 weeks old. Computing device 104 may classify age datum to neonatal progression level by generating a neonatal classification algorithm. A “neonatal classification algorithm,” as used in this disclosure is any calculation and/or series of calculations that identify to which set of categories or “bins” a new observation or input belongs. Generating neonatal classification algorithm may include generating a machine learning model using a classification algorithm. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. Computing device 104 may utilize neonatal classification model that utilizes age datum as an input and outputs neonatal progression level.

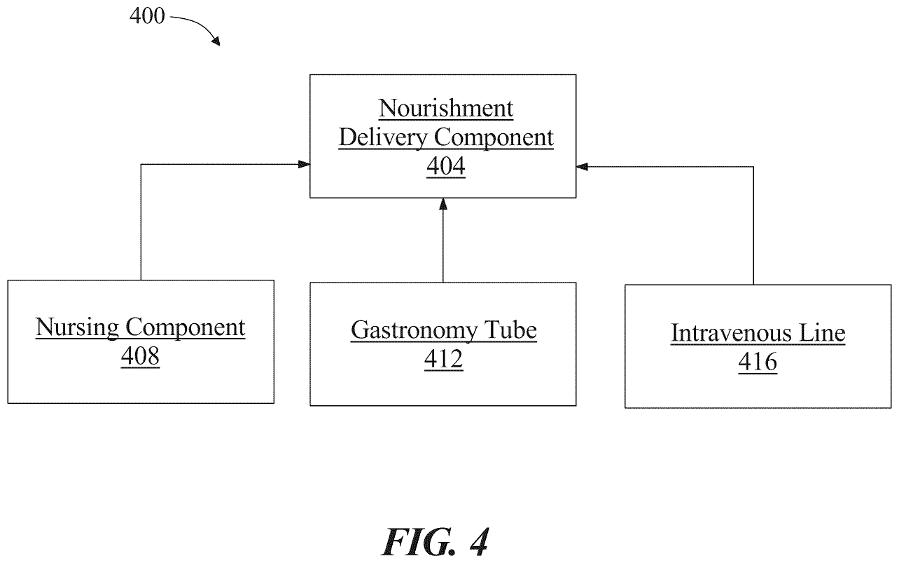

With continued reference to FIG. 1, computing device 104 may be configured to generate a neonatal phase label. A “neonatal phase label,” as used in this disclosure, is any textual, numerical, and/or symbolic data that identifies whether age datum belongs to a particular class or not, where a class may include any neonatal progression level and/or neonatal phase. For example, a neonatal phase label may indicate that current age datum belongs to a 36-week neonatal phase label and the age datum does not belong to a 28-week phase, a 1-month phase, a 6-month phase, and the like thereof. Additionally or alternatively, computing device 104 may identify a nourishment delivery component as a function of calculating neonatal phase, wherein a nourishment delivery component is a component that allows for nourishment to be received by an infant, as described below in detail, in reference to FIG. 4.

Still referring to FIG. 1, computing device 104 generates a nourishment program 140 as a function of aliment 136. As used in this disclosure a “nourishment program” is a program consisting of one or more aliments that are to be administered to an infant over a given time period, wherein a time period is a temporal measurement such as seconds, minutes, hours, days, weeks, months, years, and the like thereof. As a non-limiting example nourishment program 140 may consist of recommending a breast milk fortified with calcium for 7 days. As a further non-limiting example nourishment program 140 may recommend formula supplemented with phosphorus for a first day, formula supplemented with sodium for a second day, and breast milk for a third day. In an embodiment, nourishment program 140 may include one or more recommendations of aliments for a mother to consume to alter and/or enhance nourishment compositions of breast milk. As a non-limiting example nourishment program may include one or more recommendations of aliments for a mother to consume, such as recommending salmon to enhance vitamin C and docosahexaenoic acid concentrations in breast milk for the infant. As a further non-limiting example, nourishment program 140 may recommend one or more diet programs such as paleo, keto, vegan, vegetarian, and the like thereof to the mother that is breastfeeding the infant.

In an embodiment, and still referring to FIG. 1, computing device 104 may develop nourishment program 140 as a function of a neonatal outcome. As used in this disclosure a “neonatal outcome” is an outcome that an aliment may generate according to a predicted and/or purposeful plan. As a non-limiting example, neonatal outcome may include a treatment outcome. As used in this disclosure a “treatment outcome” is an intended outcome that is designed to at least reverse and/or eliminate neonatal indicator element 108 associated with neonatal profile 116 and/or neonatal disorder. As a non-limiting example, a treatment outcome may include reversing the effects of the neonatal disorder jaundice. As a further non-limiting example, a treatment outcome includes reversing the neonatal disorder of hydrocephalus. Neonatal outcome may include a prevention outcome. As used in this disclosure a “prevention outcome” is an intended outcome that is designed to at least prevent and/or avert neonatal indicator element 108 associated with neonatal profile 116 and/or neonatal disorder. As a non-limiting example, a prevention outcome may include preventing the development of the neonatal disorder of bradycardia.

Still referring to FIG. 1, computing device 104 may develop nourishment program 140 as a function of aliment 136 and treatment outcome using a nourishment machine-learning model. As used in this disclosure a “nourishment machine-learning model” is a machine-learning model to produce a nourishment program output given aliments and/or neonatal outcomes as inputs; this is in contrast to a non-machine learning software program where the commands to be executed are determined in advance by a user and written in a programming language. Nourishment machine-learning model may include one or more nourishment machine-learning processes such as supervised, unsupervised, or reinforcement machine-learning processes that computing device 104 and/or a remote device may or may not use in the development of nourishment program 140. Nourishment machine-learning process may include, without limitation machine learning processes such as simple linear regression, multiple linear regression, polynomial regression, support vector regression, ridge regression, lasso regression, elasticnet regression, decision tree regression, random forest regression, logistic regression, logistic classification, K-nearest neighbors, support vector machines, kernel support vector machines, naïve bayes, decision tree classification, random forest classification, K-means clustering, hierarchical clustering, dimensionality reduction, principal component analysis, linear discriminant analysis, kernel principal component analysis, Q-learning, State Action Reward State Action (SARSA), Deep-Q network, Markov decision processes, Deep Deterministic Policy Gradient (DDPG), or the like thereof.

Still referring to FIG. 1, computing device 104 may train nourishment machine-learning process as a function of a nourishment training set. As used in this disclosure a “nourishment training set” is a training set that correlates a neonatal outcome to an aliment. The nourishment training set may be received as a function of user-entered aliments, neonatal outcomes, and/or nourishment programs. For example, and without limitation, a neonatal outcome of treating anemia may correlate to an aliment of iron. Computing device 104 may receive nourishment training by receiving correlations of neonatal outcomes and/or aliments that were previously received and/or determined during a previous iteration of developing nourishment programs. The nourishment training set may be received by one or more remote devices that at least correlate a neonatal outcome and/or aliment to a nourishment program, wherein a remote device is an external device to computing device 104, as described above. Nourishment training set may be received in the form of one or more user-entered correlations of a neonatal outcome and/or aliment to a nourishment program. Additionally or alternatively, a user may include an informed advisor, wherein an informed advisor may include, without limitation, neonatologists, pediatricians, family physicians, and the like thereof.

Still referring to FIG. 1, computing device 104 may receive nourishment machine-learning model from the remote device that utilizes one or more nourishment machine learning processes, wherein a remote device is described above in detail. For example, and without limitation, a remote device may include a computing device, external device, processor, and the like thereof. The remote device may perform the nourishment machine-learning process using the nourishment training set to develop nourishment program 140 and transmit the output to computing device 104. The remote device may transmit a signal, bit, datum, or parameter to computing device 104 that at least relates to nourishment program 140. Additionally or alternatively, the remote device may provide an updated machine-learning model. For example, and without limitation, an updated machine-learning model may be comprised of a firmware update, a software update, a nourishment machine-learning process correction, and the like thereof. As a non-limiting example a software update may incorporate a new neonatal outcome that relates to a modified aliment. Additionally or alternatively, the updated machine learning model may be transmitted to the remote device, wherein the remote device may replace the nourishment machine-learning model with the updated machine-learning model and develop the nourishment program as a function of the neonatal outcome using the updated machine-learning model. The updated machine-learning model may be transmitted by the remote device and received by computing device 104 as a software update, firmware update, or corrected nourishment machine-learning model. For example, and without limitation nourishment machine-learning model may utilize a neural net machine-learning process, wherein the updated machine-learning model may incorporate decision tree machine-learning processes.

With continued reference to FIG. 1, computing device 104 may generate nourishment program 140 using an LLM. Computing device 104 may include a large language model (LLM). A “large language model,” as used herein, is a deep learning data structure that can recognize, summarize, translate, predict and/or generate text and other content based on knowledge gained from massive datasets. Large language models may be trained on large sets of data. Training sets may be drawn from diverse sets of data such as, as non-limiting examples, novels, blog posts, articles, emails, unstructured data, electronic records, and the like. In some embodiments, training sets may include a variety of subject matters, such as, as nonlimiting examples, medical report documents, electronic health records, entity documents, business documents, inventory documentation, emails, user communications, advertising documents, newspaper articles, past examples of nourishment program, doctor communications, scholarly articles, and the like. In some embodiments, training sets of an LLM may include information from one or more public or private databases. As a non-limiting example, training sets may include databases associated with an entity. In some embodiments, training sets may include portions of documents associated with the electronic records correlated to examples of outputs. In an embodiment, an LLM may include one or more architectures for based on capability requirements of an LLM. Exemplary architectures may include, without limitation, GPT (Generative Pretrained Transformer), BERT (Bidirectional Encoder Representations from Transformers), T5 (Text-To-Text Transfer Transformer), and the like. Architecture choice may depend on a needed capability such generative, contextual, or other specific capabilities.

With continued reference to FIG. 1, in some embodiments, an LLM may be generally trained. As used in this disclosure, a “generally trained” LLM is an LLM that is trained on a general training set comprising a variety of subject matters, data sets, and fields. In some embodiments, an LLM may be initially generally trained. Additionally, or alternatively, an LLM may be specifically trained. As used in this disclosure, a “specifically trained” LLM is an LLM that is trained on a specific training set, wherein the specific training set includes data including specific correlations for the LLM to learn. As a non-limiting example, an LLM may be generally trained on a general training set, then specifically trained on a specific training set. In an embodiment, specific training of an LLM may be performed using a supervised machine learning process. In some embodiments, generally training an LLM may be performed using an unsupervised machine learning process. As a non-limiting example, specific training set may include information from a database. As a non-limiting example, specific training set may include text related to the users such as user specific data for electronic records correlated to examples of outputs. In an embodiment, training one or more machine learning models may include setting the parameters of the one or more models (weights and biases) either randomly or using a pretrained model. Generally training one or more machine learning models on a large corpus of text data can provide a starting point for fine-tuning on a specific task. A model such as an LLM may learn by adjusting its parameters during the training process to minimize a defined loss function, which measures the difference between predicted outputs and ground truth. Once a model has been generally trained, the model may then be specifically trained to fine-tune the pretrained model on task-specific data to adapt it to the target task. Fine-tuning may involve training a model with task-specific training data, adjusting the model's weights to optimize performance for the particular task. In some cases, this may include optimizing the model's performance by fine-tuning hyperparameters such as learning rate, batch size, and regularization. Hyperparameter tuning may help in achieving the best performance and convergence during training. In an embodiment, fine-tuning a pretrained model such as an LLM may include fine-tuning the pretrained model using Low-Rank Adaptation (LoRA). As used in this disclosure, “Low-Rank Adaptation” is a training technique for large language models that modifies a subset of parameters in the model. Low-Rank Adaptation may be configured to make the training process more computationally efficient by avoiding a need to train an entire model from scratch. In an exemplary embodiment, a subset of parameters that are updated may include parameters that are associated with a specific task or domain.

With continued reference to FIG. 1, in some embodiments an LLM may include and/or be produced using Generative Pretrained Transformer (GPT), GPT-2, GPT-3, GPT-4, and the like. GPT, GPT-2, GPT-3, GPT-3.5, and GPT-4 are products of Open AI Inc., of San Francisco, CA. An LLM may include a text prediction based algorithm configured to receive an article and apply a probability distribution to the words already typed in a sentence to work out the most likely word to come next in augmented articles. For example, if some words that have already been typed are “Nice to meet”, then it may be highly likely that the word “you” will come next. An LLM may output such predictions by ranking words by likelihood or a prompt parameter. For the example given above, an LLM may score “you” as the most likely, “your” as the next most likely, “his” or “her” next, and the like. An LLM may include an encoder component and a decoder component.

Still referring to FIG. 1, an LLM may include a transformer architecture. In some embodiments, encoder component of an LLM may include transformer architecture. A “transformer architecture,” for the purposes of this disclosure is a neural network architecture that uses self-attention and positional encoding. Transformer architecture may be designed to process sequential input data, such as natural language, with applications towards tasks such as translation and text summarization. Transformer architecture may process the entire input all at once. “Positional encoding,” for the purposes of this disclosure, refers to a data processing technique that encodes the location or position of an entity in a sequence. In some embodiments, each position in the sequence may be assigned a unique representation. In some embodiments, positional encoding may include mapping each position in the sequence to a position vector. In some embodiments, trigonometric functions, such as sine and cosine, may be used to determine the values in the position vector. In some embodiments, position vectors for a plurality of positions in a sequence may be assembled into a position matrix, wherein each row of position matrix may represent a position in the sequence.

With continued reference to FIG. 1, an LLM and/or transformer architecture may include an attention mechanism. An “attention mechanism,” as used herein, is a part of a neural architecture that enables a system to dynamically quantify the relevant features of the input data. In the case of natural language processing, input data may be a sequence of textual elements. It may be applied directly to the raw input or to its higher-level representation.

With continued reference to FIG. 1, attention mechanism may represent an improvement over a limitation of an encoder-decoder model. An encoder-decider model encodes an input sequence to one fixed length vector from which the output is decoded at each time step. This issue may be seen as a problem when decoding long sequences because it may make it difficult for the neural network to cope with long sentences, such as those that are longer than the sentences in the training corpus. Applying an attention mechanism, an LLM may predict the next word by searching for a set of positions in a source sentence where the most relevant information is concentrated. An LLM may then predict the next word based on context vectors associated with these source positions and all the previously generated target words, such as textual data of a dictionary correlated to a prompt in a training data set. A “context vector,” as used herein, are fixed-length vector representations useful for document retrieval and word sense disambiguation.

Still referring to FIG. 1, attention mechanism may include, without limitation, generalized attention self-attention, multi-head attention, additive attention, global attention, and the like. In generalized attention, when a sequence of words or an image is fed to an LLM, it may verify each element of the input sequence and compare it against the output sequence. Each iteration may involve the mechanism's encoder capturing the input sequence and comparing it with each element of the decoder's sequence. From the comparison scores, the mechanism may then select the words or parts of the image that it needs to pay attention to. In self-attention, an LLM may pick up particular parts at different positions in the input sequence and over time compute an initial composition of the output sequence. In multi-head attention, an LLM may include a transformer model of an attention mechanism. Attention mechanisms, as described above, may provide context for any position in the input sequence. For example, if the input data is a natural language sentence, the transformer does not have to process one word at a time. In multi-head attention, computations by an LLM may be repeated over several iterations, each computation may form parallel layers known as attention heads. Each separate head may independently pass the input sequence and corresponding output sequence clement through a separate head. A final attention score may be produced by combining attention scores at each head so that every nuance of the input sequence is taken into consideration. In additive attention (Bahdanau attention mechanism), an LLM may make use of attention alignment scores based on a number of factors. Alignment scores may be calculated at different points in a neural network, and/or at different stages represented by discrete neural networks. Source or input sequence words are correlated with target or output sequence words but not to an exact degree. This correlation may take into account all hidden states and the final alignment score is the summation of the matrix of alignment scores. In global attention (Luong mechanism), in situations where neural machine translations are required, an LLM may either attend to all source words or predict the target sentence, thereby attending to a smaller subset of words.

With continued reference to FIG. 1, multi-headed attention in encoder may apply a specific attention mechanism called self-attention. Self-attention allows models such as an LLM or components thereof to associate each word in the input, to other words. As a non-limiting example, an LLM may learn to associate the word “you”, with “how” and “are”. It's also possible that an LLM learns that words structured in this pattern are typically a question and to respond appropriately. In some embodiments, to achieve self-attention, input may be fed into three distinct fully connected neural network layers to create query, key, and value vectors. Query, key, and value vectors may be fed through a linear layer; then, the query and key vectors may be multiplied using dot product matrix multiplication in order to produce a score matrix. The score matrix may determine the amount of focus for a word should be put on other words (thus, each word may be a score that corresponds to other words in the time-step). The values in score matrix may be scaled down. As a non-limiting example, score matrix may be divided by the square root of the dimension of the query and key vectors. In some embodiments, the softmax of the scaled scores in score matrix may be taken. The output of this softmax function may be called the attention weights. Attention weights may be multiplied by your value vector to obtain an output vector. The output vector may then be fed through a final linear layer.

Still referencing FIG. 1, in order to use self-attention in a multi-headed attention computation, query, key, and value may be split into N vectors before applying self-attention. Each self-attention process may be called a “head.” Each head may produce an output vector and each output vector from each head may be concatenated into a single vector. This single vector may then be fed through the final linear layer discussed above. In theory, each head can learn something different from the input, therefore giving the encoder model more representation power.

With continued reference to FIG. 1, encoder of transformer may include a residual connection. Residual connection may include adding the output from multi-headed attention to the positional input embedding. In some embodiments, the output from residual connection may go through a layer normalization. In some embodiments, the normalized residual output may be projected through a pointwise feed-forward network for further processing. The pointwise feed-forward network may include a couple of linear layers with a ReLU activation in between. The output may then be added to the input of the pointwise feed-forward network and further normalized.

Continuing to refer to FIG. 1, transformer architecture may include a decoder. Decoder may a multi-headed attention layer, a pointwise feed-forward layer, one or more residual connections, and layer normalization (particularly after each sub-layer), as discussed in more detail above. In some embodiments, decoder may include two multi-headed attention layers. In some embodiments, decoder may be autoregressive. For the purposes of this disclosure, “autoregressive” means that the decoder takes in a list of previous outputs as inputs along with encoder outputs containing attention information from the input.

With further reference to FIG. 1, in some embodiments, input to decoder may go through an embedding layer and positional encoding layer in order to obtain positional embeddings. Decoder may include a first multi-headed attention layer, wherein the first multi-headed attention layer may receive positional embeddings.

With continued reference to FIG. 1, first multi-headed attention layer may be configured to not condition to future tokens. As a non-limiting example, when computing attention scores on the word “am,” decoder should not have access to the word “fine” in “I am fine,” because that word is a future word that was generated after. The word “am” should only have access to itself and the words before it. In some embodiments, this may be accomplished by implementing a look-ahead mask. Look ahead mask is a matrix of the same dimensions as the scaled attention score matrix that is filled with “0s” and negative infinities. For example, the top right triangle portion of look-ahead mask may be filled with negative infinities. Look-ahead mask may be added to scaled attention score matrix to obtain a masked score matrix. Masked score matrix may include scaled attention scores in the lower-left triangle of the matrix and negative infinities in the upper-right triangle of the matrix. Then, when the softmax of this matrix is taken, the negative infinities will be zeroed out; this leaves zero attention scores for “future tokens.”

Still referring to FIG. 1, second multi-headed attention layer may use encoder outputs as queries and keys and the outputs from the first multi-headed attention layer as values. This process matches the encoder's input to the decoder's input, allowing the decoder to decide which encoder input is relevant to put a focus on. The output from second multi-headed attention layer may be fed through a pointwise feedforward layer for further processing.

With continued reference to FIG. 1, the output of the pointwise feedforward layer may be fed through a final linear layer. This final linear layer may act as a classifier. This classifier may be as big as the number of classes that you have. For example, if you have 10,000 classes for 10,000 words, the output of that classifier will be of size 10,000. The output of this classifier may be fed into a softmax layer which may serve to produce probability scores between zero and one. The index may be taken of the highest probability score in order to determine a predicted word.

Still referring to FIG. 1, decoder may take this output and add it to the decoder inputs. Decoder may continue decoding until a token is predicted. Decoder may stop decoding once it predicts an end token.

Continuing to refer to FIG. 1, in some embodiment, decoder may be stacked N layers high, with each layer taking in inputs from the encoder and layers before it. Stacking layers may allow an LLM to learn to extract and focus on different combinations of attention from its attention heads.

With continued reference to FIG. 1, an LLM may receive an input. Input may include a string of one or more characters. Inputs may additionally include unstructured data. For example, input may include one or more words, a sentence, a paragraph, a thought, a query, and the like. A “query” for the purposes of the disclosure is a string of characters that poses a question. In some embodiments, input may be received from a user device. User device may be any computing device that is used by a user. As non-limiting examples, user device may include desktops, laptops, smartphones, tablets, and the like. In some embodiments, input may include any set of data associated with Neonatal profile. In some embodiments, LLM may be configured to receive neonatal profile 116 (or updated neonatal profile) as input and output a nourishment program 140. In some embodiments, LLM may be configured to receive aliment 136 as input and output a nourishment program 140. In some embodiments, LLM may be configured to receive aliment 136 and neonatal profile 116 (or updated neonatal profile) as input and output a nourishment program 140.

With continued reference to FIG. 1, an LLM may generate at least one annotation as an output. At least one annotation may be any annotation as described herein. In some embodiments, an LLM may include multiple sets of transformer architecture as described above. Output may include a textual output. A “textual output,” for the purposes of this disclosure is an output comprising a string of one or more characters. Textual output may include, for example, a plurality of annotations for unstructured data. In some embodiments, textual output may include a phrase or sentence identifying the status of a user query. In some embodiments, textual output may include a sentence or plurality of sentences describing a response to a user query. As a non-limiting example, this may include restrictions, timing, advice, dangers, benefits, and the like.

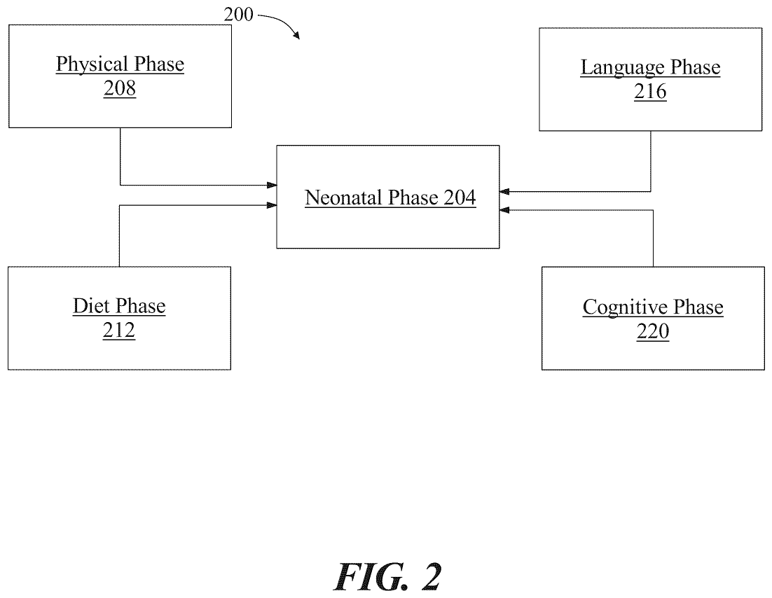

Now referring to FIG. 2, an exemplary embodiment 200 of a neonatal phase 204 is illustrated. As used in this disclosure a “neonatal phase” is a phase of development that an infant may or may not progress through. Neonatal phase 204 may include a physical phase 208. As used in this disclosure a “physical phase” is a developmental phase associated with physical milestones that an infant may or may not progress through. For example, and without limitation, physical phase 208 may include one or more actions including lifting their head, holding their head up, holding their head steady, bearing weight on their legs, playing with their hands and feet, and the like thereof. Neonatal phase 204 may include a diet phase 212. As used in this disclosure a “diet phase” is a developmental phase associated with dietary milestones that an infant may or may not progress through. For example, and without limitation, dietary phase 212 may include one or more capabilities to ingest varying aliment types and/or nutrients. In an embodiment, and without limitation, diet phase may include developing from a parenteral administration of nutrients to breast milk. Neonatal phase 204 may include a language phase 216. As used in this disclosure a “language phase” is a developmental phase associated with language milestones that an infant may or may not progress through. For example, and without limitation, language phase 216 may include one or more language milestones such as crying, gurgles, coos, laughs, imitation sounds, jabbers, and the like thereof. Neonatal phase 204 may include a cognitive phase 220. As used in this disclosure a “cognitive phase” is a developmental phase associated with cognitive milestones that an infant may or may not progress through. For example, and without limitation, cognitive phase 220 may include one or more cognitive milestones such as responses to sounds, tracking of movements with the infant's eyes, identification of objects, distinguishment of colors, and the like thereof.

Now referring to FIG. 3, an exemplary embodiment 300 of an aliment directory 304 according to an embodiment of the invention is illustrated. Aliment directory 304 may be implemented, without limitation, as a relational databank, a key-value retrieval databank such as a NOSQL databank, or any other format or structure for use as a databank that a person skilled in the art would recognize as suitable upon review of the entirety of this disclosure. Aliment directory 304 may alternatively or additionally be implemented using a distributed data storage protocol and/or data structure, such as a distributed hash table or the like. Aliment directory 304 may include a plurality of data entries and/or records as described above. Data entries in a databank may be flagged with or linked to one or more additional elements of information, which may be reflected in data entry cells and/or in linked tables such as tables related by one or more indices in a relational database. Persons skilled in the art, upon reviewing the entirety of this disclosure, will be aware of various ways in which data entries in a databank may store, retrieve, organize, and/or reflect data and/or records as used herein, as well as categories and/or populations of data consistently with this disclosure. Aliment directory 304 may include a carbohydrate tableset 308. Carbohydrate tableset 308 may relate to a nourishment composition of an aliment with respect to the quantity and/or type of carbohydrates in the aliment. As a non-limiting example, carbohydrate tableset 308 may include monosaccharides, disaccharides, oligosaccharides, polysaccharides, and the like thereof. Aliment directory 304 may include a fat tableset 312. Fat tableset 312 may relate to a nourishment composition of an aliment with respect to the quantity and/or type of esterified fatty acids in the aliment. Fat tableset 312 may include, without limitation, triglycerides, monoglycerides, diglycerides, phospholipids, sterols, waxes, and free fatty acids. Aliment directory 304 may include a fiber tableset 316. Fiber tableset 316 may relate to a nourishment composition of an aliment with respect to the quantity and/or type of fiber in the aliment. As a non-limiting example, fiber tableset 316 may include soluble fiber, such as beta-glucans, raw guar gum, psyllium, inulin, and the like thereof as well as insoluble fiber, such as wheat bran, cellulose, lignin, and the like thereof. Aliment directory 304 may include a mineral tableset 320. Mineral tableset 320 may relate to a nourishment composition of an aliment with respect to the quantity and/or type of minerals in the aliment. As a non-limiting example, mineral tableset 320 may include calcium, phosphorus, magnesium, sodium, potassium, chloride, sulfur, iron, manganese, copper, iodine, zing, cobalt, fluoride, selenium, and the like thereof. Aliment directory 304 may include a protein tableset 324. Protein tableset 324 may relate to a nourishment composition of an aliment with respect to the quantity and/or type of proteins in the aliment. As a non-limiting example, protein tableset 324 may include amino acids combinations, wherein amino acids may include, without limitation, alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, and the like thereof. Aliment directory 304 may include a vitamin tableset 328. Vitamin tableset 328 may relate to a nourishment composition of an aliment with respect to the quantity and/or type of vitamins in the aliment. As a non-limiting example, vitamin tableset 328 may include vitamin A, vitamin B₁, vitamin B₂, vitamin B₃, vitamin B₅, vitamin B₆, vitamin B₇, vitamin B₉, vitamin B₁₂, vitamin C, vitamin D, vitamin E, vitamin K, and the like thereof.

Referring now to FIG. 4, an exemplary embodiment of a nourishment delivery component 404 is illustrated. As used in this disclosure a “nourishment delivery component” is a component that allows for nourishment to be received by an infant. For example, and without limitation, nourishment delivery component may include any component that aids in delivery nutrients and/or sustenance to an infant. Nourishment delivery component 404 may include a nursing component 408. As used in this disclosure a “nursing component” is a component that allows for nourishment to be received by an infant as a function of a nursing mechanism. For example, and without limitation, nursing component 408 may include breast feeding, wet nursing, dry-nursing, and the like thereof. In an embodiment nursing component may include a nutritional recommendation. As used in this disclosure a “nutritional recommendation” is a recommendation for a nursing mother to consume a nutrient to alter and/or modify breast milk. For example, and without limitation, nutritional recommendation may include recommending soybean oil, walnuts, chia, hemp, and/or flax seeds to increase concentration of omega fatty acids in breast milk. As a further non-limiting example nutritional recommendation may include recommending removing dairy and soy products from the mother's diet to reduce the amount of CMPI in breast milk for an infant that has a CMPO aliment intolerance. As a further non-limiting example, nutritional recommendation may include recommending to a nursing mother to reduce exercise frequency to reduce the concentration of lactic acid in the breast milk. In an embodiment, and without limitation, nursing component 408 may include one or more bottle feeding techniques, wherein an aliment of a plurality of aliments are placed within a bottle that is sealed with a nipple extruding from the top of the bottle to allow an infant to suckle and/or nurse from the nipple. Nutritional recommendation may include recommending one or more formulas for a bottle-feeding technique to reduce the effects of an aliment intolerance, wherein an aliment intolerance is a difficulty digesting a particular aliment as described above, in reference to FIG. 1. For example, and without limitation, nutritional recommendation may include recommending a soy-based formula as opposed to a milk based formula to reduce the effects of a lactose intolerance. As a further non-limiting example, nutritional recommendation may include recommending an elemental amino acid-based formula as opposed to a milk-based formula to reduce the effects of a CMPI aliment intolerance. Nourishment delivery component 404 may include a gastronomy tube 412. As used in this disclosure a “gastronomy tube” is a component that allows for nourishment to be received by an infant as a function of a feeding tube. For example, and without limitation, gastronomy tube 412 may include a gavage tube, enteral feeding tube, nasogastric feeding tube, nasojejunal feeding tube, gastrojejunal feeding tube, jejunal feeding tube, and the like thereof. Nourishment delivery component 404 may include an intravenous line 416. As used in this disclosure an “intravenous line” is a component that allows for nourishment to be received by an infant as a function of a parenteral feeding mechanism. For example, and without limitation, intravenous line may include a tube in a vein located within an infant's hand, foot, scalp, belly button, and the like thereof.

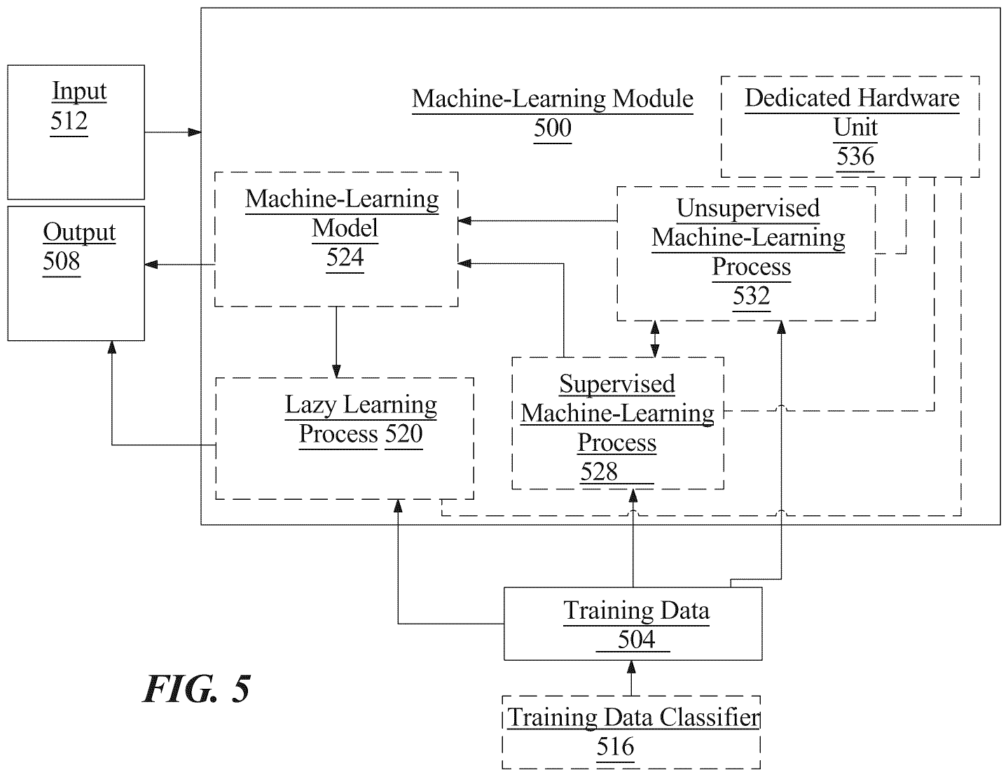

Referring now to FIG. 5, an exemplary embodiment of a machine-learning module 500 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 504 to generate an algorithm instantiated in hardware or software logic, data structures, and/or functions that will be performed by a computing device/module to produce outputs 508 given data provided as inputs 512; this is in contrast to a non-machine learning software program where the commands to be executed are determined in advance by a user and written in a programming language.

Still referring to FIG. 5, “training data,” as used herein, is data containing correlations that a machine-learning process may use to model relationships between two or more categories of data elements. For instance, and without limitation, training data 504 may include a plurality of data entries, also known as “training examples,” each entry representing a set of data elements that were recorded, received, and/or generated together; data elements may be correlated by shared existence in a given data entry, by proximity in a given data entry, or the like. Multiple data entries in training data 504 may evince one or more trends in correlations between categories of data elements; for instance, and without limitation, a higher value of a first data element belonging to a first category of data clement may tend to correlate to a higher value of a second data element belonging to a second category of data clement, indicating a possible proportional or other mathematical relationship linking values belonging to the two categories. Multiple categories of data elements may be related in training data 504 according to various correlations; correlations may indicate causative and/or predictive links between categories of data elements, which may be modeled as relationships such as mathematical relationships by machine-learning processes as described in further detail below. Training data 504 may be formatted and/or organized by categories of data elements, for instance by associating data elements with one or more descriptors corresponding to categories of data elements. As a non-limiting example, training data 504 may include data entered in standardized forms by persons or processes, such that entry of a given data element in a given field in a form may be mapped to one or more descriptors of categories. Elements in training data 504 may be linked to descriptors of categories by tags, tokens, or other data elements; for instance, and without limitation, training data 504 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 5, training data 504 may include one or more elements that are not categorized; that is, training data 504 may not be formatted or contain descriptors for some elements of data. Machine-learning algorithms and/or other processes may sort training data 504 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 504 to be made applicable for two or more distinct machine-learning algorithms as described in further detail below. Training data 504 used by machine-learning module 500 may correlate any input data as described in this disclosure to any output data as described in this disclosure. As a non-limiting illustrative example training data may include waste-related photos correlated to neonatal disorders or infant sensor measurements/infant measurements correlated to patterns.

Further referring to FIG. 5, training data may be filtered, sorted, and/or selected using one or more supervised and/or unsupervised machine-learning processes and/or models as described in further detail below; such models may include without limitation a training data classifier 516. Training data classifier 516 may include a “classifier,” which as used in this disclosure is a machine-learning learning model as defined below, such as a data structure representing and/or using a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. A distance metric may include any norm, such as, without limitation, a Pythagorean norm. Machine-learning module 500 may generate a classifier using a classification algorithm, defined as a processes whereby a computing device and/or any module and/or component operating thereon derives a classifier from training data 504. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 516 may classify elements of training data to [something that characterizes a sub-population, such as a cohort of persons and/or other analyzed items and/or phenomena for which a subset of training data may be selected].

Still referring to FIG. 5, computing device 504 may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular clement is independent of the value of any other element, given a class variable. Naïve Bayes classification algorithm may be based on Bayes Theorem expressed as P(A/B)=P(B/A) P(A)÷P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Computing device 504 may then calculate a likelihood table by calculating probabilities of different data entries and classification labels. Computing device 504 may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 5, computing device 504 may be configured to generate a classifier using a K-nearest neighbors (KNN) algorithm. A “K-nearest neighbors algorithm” as used in this disclosure, includes a classification method that utilizes feature similarity to analyze how closely out-of-sample- features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 5, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least two values. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute l as derived using a Pythagorean norm: l=\sqrt{Σᵢ₌₀ⁿaᵢ²}, where aᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

With further reference to FIG. 5, training examples for use as training data may be selected from a population of potential examples according to cohorts relevant to an analytical problem to be solved, a classification task, or the like. Alternatively or additionally, training data may be selected to span a set of likely circumstances or inputs for a machine-learning model and/or process to encounter when deployed. For instance, and without limitation, for each category of input data to a machine-learning process or model that may exist in a range of values in a population of phenomena such as images, user data, process data, physical data, or the like, a computing device, processor, and/or machine-learning model may select training examples representing each possible value on such a range and/or a representative sample of values on such a range. Selection of a representative sample may include selection of training examples in proportions matching a statistically determined and/or predicted distribution of such values according to relative frequency, such that, for instance, values encountered more frequently in a population of data so analyzed are represented by more training examples than values that are encountered less frequently. Alternatively or additionally, a set of training examples may be compared to a collection of representative values in a database and/or presented to a user, so that a process can detect, automatically or via user input, one or more values that are not included in the set of training examples. Computing device, processor, and/or module may automatically generate a missing training example; this may be done by receiving and/or retrieving a missing input and/or output value and correlating the missing input and/or output value with a corresponding output and/or input value collocated in a data record with the retrieved value, provided by a user and/or other device, or the like.

Continuing to refer to FIG. 5, computer, processor, and/or module may be configured to preprocess training data. “Preprocessing” training data, as used in this disclosure, is transforming training data from raw form to a format that can be used for training a machine learning model. Preprocessing may include sanitizing, feature selection, feature scaling, data augmentation and the like.

Still referring to FIG. 5, computer, processor, and/or module may be configured to sanitize training data. “Sanitizing” training data, as used in this disclosure, is a process whereby training examples are removed that interfere with convergence of a machine-learning model and/or process to a useful result. For instance, and without limitation, a training example may include an input and/or output value that is an outlier from typically encountered values, such that a machine-learning algorithm using the training example will be adapted to an unlikely amount as an input and/or output; a value that is more than a threshold number of standard deviations away from an average, mean, or expected value, for instance, may be eliminated. Alternatively or additionally, one or more training examples may be identified as having poor quality data, where “poor quality” is defined as having a signal to noise ratio below a threshold value. Sanitizing may include steps such as removing duplicative or otherwise redundant data, interpolating missing data, correcting data errors, standardizing data, identifying outliers, and the like. In a nonlimiting example, santization may include utilizing algorithms for identifying duplicate entries or spell-check algorithms.

As a non-limiting example, and with further reference to FIG. 5, images used to train an image classifier or other machine-learning model and/or process that takes images as inputs or generates images as outputs may be rejected if image quality is below a threshold value. For instance, and without limitation, computing device, processor, and/or module may perform blur detection, and eliminate one or more Blur detection may be performed, as a non-limiting example, by taking Fourier transform, or an approximation such as a Fast Fourier Transform (FFT) of the image and analyzing a distribution of low and high frequencies in the resulting frequency-domain depiction of the image; numbers of high-frequency values below a threshold level may indicate blurriness. As a further non-limiting example, detection of blurriness may be performed by convolving an image, a channel of an image, or the like with a Laplacian kernel; this may generate a numerical score reflecting a number of rapid changes in intensity shown in the image, such that a high score indicates clarity and a low score indicates blurriness. Blurriness detection may be performed using a gradient-based operator, which measures operators based on the gradient or first derivative of an image, based on the hypothesis that rapid changes indicate sharp edges in the image, and thus are indicative of a lower degree of blurriness. Blur detection may be performed using Wavelet-based operator, which takes advantage of the capability of coefficients of the discrete wavelet transform to describe the frequency and spatial content of images. Blur detection may be performed using statistics-based operators take advantage of several image statistics as texture descriptors in order to compute a focus level. Blur detection may be performed by using discrete cosine transform (DCT) coefficients in order to compute a focus level of an image from its frequency content.

Continuing to refer to FIG. 5, computing device, processor, and/or module may be configured to precondition one or more training examples. For instance, and without limitation, where a machine learning model and/or process has one or more inputs and/or outputs requiring, transmitting, or receiving a certain number of bits, samples, or other units of data, one or more training examples' elements to be used as or compared to inputs and/or outputs may be modified to have such a number of units of data. For instance, a computing device, processor, and/or module may convert a smaller number of units, such as in a low pixel count image, into a desired number of units, for instance by upsampling and interpolating. As a non-limiting example, a low pixel count image may have 100 pixels, however a desired number of pixels may be 128. Processor may interpolate the low pixel count image to convert the 100 pixels into 128 pixels. It should also be noted that one of ordinary skill in the art, upon reading this disclosure, would know the various methods to interpolate a smaller number of data units such as samples, pixels, bits, or the like to a desired number of such units. In some instances, a set of interpolation rules may be trained by sets of highly detailed inputs and/or outputs and corresponding inputs and/or outputs downsampled to smaller numbers of units, and a neural network or other machine learning model that is trained to predict interpolated pixel values using the training data. As a non-limiting example, a sample input and/or output, such as a sample picture, with sample-expanded data units (e.g., pixels added between the original pixels) may be input to a neural network or machine-learning model and output a pseudo replica sample-picture with dummy values assigned to pixels between the original pixels based on a set of interpolation rules. As a non-limiting example, in the context of an image classifier, a machine-learning model may have a set of interpolation rules trained by sets of highly detailed images and images that have been downsampled to smaller numbers of pixels, and a neural network or other machine learning model that is trained using those examples to predict interpolated pixel values in a facial picture context. As a result, an input with sample-expanded data units (the ones added between the original data units, with dummy values) may be run through a trained neural network and/or model, which may fill in values to replace the dummy values. Alternatively or additionally, processor, computing device, and/or module may utilize sample expander methods, a low-pass filter, or both. As used in this disclosure, a “low-pass filter” is a filter that passes signals with a frequency lower than a selected cutoff frequency and attenuates signals with frequencies higher than the cutoff frequency. The exact frequency response of the filter depends on the filter design. Computing device, processor, and/or module may use averaging, such as luma or chroma averaging in images, to fill in data units in between original data units.

In some embodiments, and with continued reference to FIG. 5, computing device, processor, and/or module may down-sample elements of a training example to a desired lower number of data elements. As a non-limiting example, a high pixel count image may have 256 pixels, however a desired number of pixels may be 128. Processor may down-sample the high pixel count image to convert the 256 pixels into 128 pixels. In some embodiments, processor may be configured to perform downsampling on data. Downsampling, also known as decimation, may include removing every Nth entry in a sequence of samples, all but every Nth entry, or the like, which is a process known as “compression,” and may be performed, for instance by an N-sample compressor implemented using hardware or software. Anti-aliasing and/or anti-imaging filters, and/or low-pass filters, may be used to clean up side-effects of compression.

Further referring to FIG. 5, feature selection includes narrowing and/or filtering training data to exclude features and/or elements, or training data including such elements, that are not relevant to a purpose for which a trained machine-learning model and/or algorithm is being trained, and/or collection of features and/or elements, or training data including such elements, on the basis of relevance or utility for an intended task or purpose for a trained machine-learning model and/or algorithm is being trained. Feature selection may be implemented, without limitation, using any process described in this disclosure, including without limitation using training data classifiers, exclusion of outliers, or the like.

With continued reference to FIG. 5, feature scaling may include, without limitation, normalization of data entries, which may be accomplished by dividing numerical fields by norms thereof, for instance as performed for vector normalization. Feature scaling may include absolute maximum scaling, wherein each quantitative datum is divided by the maximum absolute value of all quantitative data of a set or subset of quantitative data. Feature scaling may include min-max scaling, in which each value X has a minimum value Xₘᵢₙ in a set or subset of values subtracted therefrom, with the result divided by the range of the values, give maximum value in the set or subset Xₘₐₓ:

$${X}_{n e w} = \frac{X - {X}_{min}}{{X}_{max} - {X}_{min}} .$$

Feature scaling may include mean normalization, which involves use of a mean value of a set and/or subset of values, Xₘₑₐₙ with maximum and minimum values:

$${X}_{n e w} = \frac{X - {X}_{mean}}{{X}_{max} - {X}_{min}} .$$

Feature scaling may include standardization, where a difference between X and Xₘₑₐₙ is divided by a standard deviation σ of a set or subset of values:

$${X}_{n e w} = \frac{X - {X}_{m e a n}}{σ} .$$

Scaling may be performed using a median value of a a set or subset X_{median} and/or interquartile range (IQR), which represents the difference between the 25^{th} percentile value and the 50^{th} percentile value (or closest values thereto by a rounding protocol), such as:

$${X}_{n e w} = \frac{X - {X}_{m e d i a n}}{IQR} .$$

Persons skilled in the art, upon reviewing the entirety of this disclosure, will be aware of various alternative or additional approaches that may be used for feature scaling.

Further referring to FIG. 5, computing device, processor, and/or module may be configured to perform one or more processes of data augmentation. “Data augmentation” as used in this disclosure is addition of data to a training set using elements and/or entries already in the dataset. Data augmentation may be accomplished, without limitation, using interpolation, generation of modified copies of existing entries and/or examples, and/or one or more generative AI processes, for instance using deep neural networks and/or generative adversarial networks; generative processes may be referred to alternatively in this context as “data synthesis” and as creating “synthetic data.” Augmentation may include performing one or more transformations on data, such as geometric, color space, affine, brightness, cropping, and/or contrast transformations of images.

Still referring to FIG. 5, machine-learning module 500 may be configured to perform a lazy-learning process 520 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 504. Heuristic may include selecting some number of highest-ranking associations and/or training data 504 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 5, machine-learning processes as described in this disclosure may be used to generate machine-learning models 524. A “machine-learning model,” as used in this disclosure, is a data structure representing and/or instantiating a mathematical and/or algorithmic representation of a relationship between inputs and outputs, as generated using any machine-learning process including without limitation any process as described above, and stored in memory; an input is submitted to a machine-learning model 524 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 524 may be generated by creating an artificial neural network, such as a convolutional neural network comprising an input layer of nodes, one or more intermediate layers, and an output layer of nodes. Connections between nodes may be created via the process of “training” the network, in which elements from a training data 504 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 5, machine-learning algorithms may include at least a supervised machine-learning process 528. At least a supervised machine-learning process 528, as defined herein, include algorithms that receive a training set relating a number of inputs to a number of outputs, and seek to generate one or more data structures representing and/or instantiating one or more mathematical relations relating inputs to outputs, where each of the one or more mathematical relations is optimal according to some criterion specified to the algorithm using some scoring function. For instance, a supervised learning algorithm may include inputs as described above as inputs, outputs as described above as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 504. Persons skilled in the art, upon reviewing the entirety of this disclosure, will be aware of various possible variations of at least a supervised machine-learning process 528 that may be used to determine relation between inputs and outputs. Supervised machine-learning processes may include classification algorithms as defined above.

With further reference to FIG. 5, training a supervised machine-learning process may include, without limitation, iteratively updating coefficients, biases, weights based on an error function, expected loss, and/or risk function. For instance, an output generated by a supervised machine-learning model using an input example in a training example may be compared to an output example from the training example; an error function may be generated based on the comparison, which may include any error function suitable for use with any machine-learning algorithm described in this disclosure, including a square of a difference between one or more sets of compared values or the like. Such an error function may be used in turn to update one or more weights, biases, coefficients, or other parameters of a machine-learning model through any suitable process including without limitation gradient descent processes, least-squares processes, and/or other processes described in this disclosure. This may be done iteratively and/or recursively to gradually tune such weights, biases, coefficients, or other parameters. Updating may be performed, in neural networks, using one or more back-propagation algorithms. Iterative and/or recursive updates to weights, biases, coefficients, or other parameters as described above may be performed until currently available training data is exhausted and/or until a convergence test is passed, where a “convergence test” is a test for a condition selected as indicating that a model and/or weights, biases, coefficients, or other parameters thereof has reached a degree of accuracy. A convergence test may, for instance, compare a difference between two or more successive errors or error function values, where differences below a threshold amount may be taken to indicate convergence. Alternatively or additionally, one or more errors and/or error function values evaluated in training iterations may be compared to a threshold.

Still referring to FIG. 5, a computing device, processor, and/or module may be configured to perform method, method step, sequence of method steps and/or algorithm described in reference to this figure, in any order and with any degree of repetition. For instance, a computing device, processor, and/or module may be configured to perform a single step, sequence and/or algorithm repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. A computing device, processor, and/or module may perform any step, sequence of steps, or algorithm in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Further referring to FIG. 5, machine learning processes may include at least an unsupervised machine-learning processes 532. An unsupervised machine-learning process, as used herein, is a process that derives inferences in datasets without regard to labels; as a result, an unsupervised machine-learning process may be free to discover any structure, relationship, and/or correlation provided in the data. Unsupervised processes 532 may not require a response variable; unsupervised processes 532 may be used to find interesting patterns and/or inferences between variables, to determine a degree of correlation between two or more variables, or the like.

Still referring to FIG. 5, machine-learning module 500 may be designed and configured to create a machine-learning model 524 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the clastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 5, machine-learning algorithms may include, without limitation, linear discriminant analysis. Machine-learning algorithm may include quadratic discriminant analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized trees, AdaBoost, gradient tree boosting, and/or voting classifier methods. Machine-learning algorithms may include neural net algorithms, including convolutional neural net processes.

Still referring to FIG. 5, a machine-learning model and/or process may be deployed or instantiated by incorporation into a program, apparatus, system and/or module. For instance, and without limitation, a machine-learning model, neural network, and/or some or all parameters thereof may be stored and/or deployed in any memory or circuitry. Parameters such as coefficients, weights, and/or biases may be stored as circuit-based constants, such as arrays of wires and/or binary inputs and/or outputs set at logic “1” and “0” voltage levels in a logic circuit to represent a number according to any suitable encoding system including twos complement or the like or may be stored in any volatile and/or non-volatile memory. Similarly, mathematical operations and input and/or output of data to or from models, neural network layers, or the like may be instantiated in hardware circuitry and/or in the form of instructions in firmware, machine-code such as binary operation code instructions, assembly language, or any higher-order programming language. Any technology for hardware and/or software instantiation of memory, instructions, data structures, and/or algorithms may be used to instantiate a machine-learning process and/or model, including without limitation any combination of production and/or configuration of non-reconfigurable hardware elements, circuits, and/or modules such as without limitation ASICs, production and/or configuration of reconfigurable hardware elements, circuits, and/or modules such as without limitation FPGAs, production and/or of non-reconfigurable and/or configuration non-rewritable memory elements, circuits, and/or modules such as without limitation non-rewritable ROM, production and/or configuration of reconfigurable and/or rewritable memory elements, circuits, and/or modules such as without limitation rewritable ROM or other memory technology described in this disclosure, and/or production and/or configuration of any computing device and/or component thereof as described in this disclosure. Such deployed and/or instantiated machine-learning model and/or algorithm may receive inputs from any other process, module, and/or component described in this disclosure, and produce outputs to any other process, module, and/or component described in this disclosure.

Continuing to refer to FIG. 5, any process of training, retraining, deployment, and/or instantiation of any machine-learning model and/or algorithm may be performed and/or repeated after an initial deployment and/or instantiation to correct, refine, and/or improve the machine-learning model and/or algorithm. Such retraining, deployment, and/or instantiation may be performed as a periodic or regular process, such as retraining, deployment, and/or instantiation at regular elapsed time periods, after some measure of volume such as a number of bytes or other measures of data processed, a number of uses or performances of processes described in this disclosure, or the like, and/or according to a software, firmware, or other update schedule. Alternatively or additionally, retraining, deployment, and/or instantiation may be event-based, and may be triggered, without limitation, by user inputs indicating sub-optimal or otherwise problematic performance and/or by automated field testing and/or auditing processes, which may compare outputs of machine-learning models and/or algorithms, and/or errors and/or error functions thereof, to any thresholds, convergence tests, or the like, and/or may compare outputs of processes described herein to similar thresholds, convergence tests or the like. Event-based retraining, deployment, and/or instantiation may alternatively or additionally be triggered by receipt and/or generation of one or more new training examples; a number of new training examples may be compared to a preconfigured threshold, where exceeding the preconfigured threshold may trigger retraining, deployment, and/or instantiation.

Still referring to FIG. 5, retraining and/or additional training may be performed using any process for training described above, using any currently or previously deployed version of a machine-learning model and/or algorithm as a starting point. Training data for retraining may be collected, preconditioned, sorted, classified, sanitized or otherwise processed according to any process described in this disclosure. Training data may include, without limitation, training examples including inputs and correlated outputs used, received, and/or generated from any version of any system, module, machine-learning model or algorithm, apparatus, and/or method described in this disclosure; such examples may be modified and/or labeled according to user feedback or other processes to indicate desired results, and/or may have actual or measured results from a process being modeled and/or predicted by system, module, machine-learning model or algorithm, apparatus, and/or method as “desired” results to be compared to outputs for training processes as described above.

Redeployment may be performed using any reconfiguring and/or rewriting of reconfigurable and/or rewritable circuit and/or memory elements; alternatively, redeployment may be performed by production of new hardware and/or software components, circuits, instructions, or the like, which may be added to and/or may replace existing hardware and/or software components, circuits, instructions, or the like.

Further referring to FIG. 5, one or more processes or algorithms described above may be performed by at least a dedicated hardware unit 536. A “dedicated hardware unit,” for the purposes of this figure, is a hardware component, circuit, or the like, aside from a principal control circuit and/or processor performing method steps as described in this disclosure, that is specifically designated or selected to perform one or more specific tasks and/or processes described in reference to this figure, such as without limitation preconditioning and/or sanitization of training data and/or training a machine-learning algorithm and/or model. A dedicated hardware unit 536 may include, without limitation, a hardware unit that can perform iterative or massed calculations, such as matrix-based calculations to update or tune parameters, weights, coefficients, and/or biases of machine-learning models and/or neural networks, efficiently using pipelining, parallel processing, or the like; such a hardware unit may be optimized for such processes by, for instance, including dedicated circuitry for matrix and/or signal processing operations that includes, e.g., multiple arithmetic and/or logical circuit units such as multipliers and/or adders that can act simultaneously and/or in parallel or the like. Such dedicated hardware units 536 may include, without limitation, graphical processing units (GPUs), dedicated signal processing modules, FPGA or other reconfigurable hardware that has been configured to instantiate parallel processing units for one or more specific tasks, or the like, A computing device, processor, apparatus, or module may be configured to instruct one or more dedicated hardware units 536 to perform one or more operations described herein, such as evaluation of model and/or algorithm outputs, one-time or iterative updates to parameters, coefficients, weights, and/or biases, and/or any other operations such as vector and/or matrix operations as described in this disclosure.

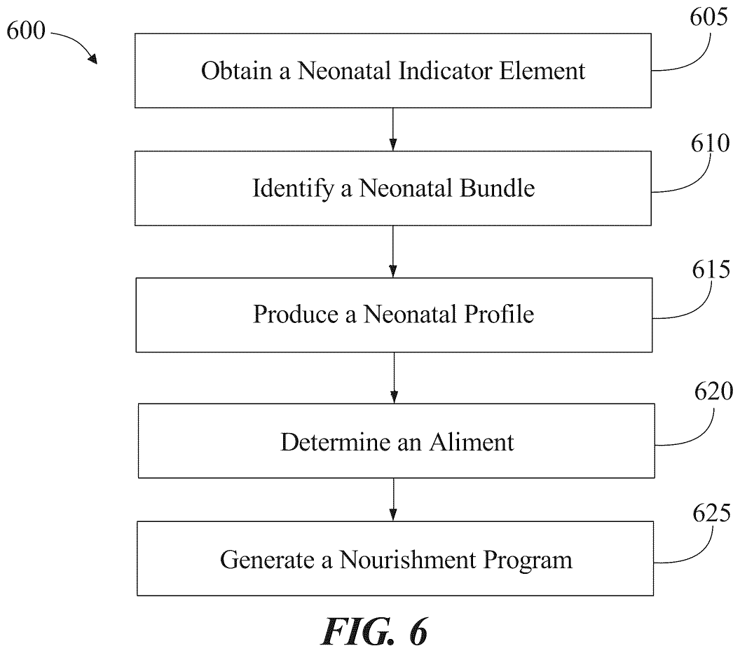

Now referring to FIG. 6, an exemplary embodiment of a method 600 for generating a neonatal disorder nourishment program is illustrated. At step 605, a computing device 104 obtains a neonatal indicator clement 108. Computing device 104 includes any of the computing device 104 as described above, in reference to FIGS. 1-5. Neonatal indicator clement 108 includes any of the neonatal indicator element 108 as described above, in reference to FIGS. 1-5.

Still referring to FIG. 6, at step 610, computing device 104 identifies a neonatal bundle 112 as a function of neonatal indicator element 108. Neonatal bundle 112 includes any of the neonatal bundle 112 as described above, in reference to FIGS. 1-5.

Still referring to FIG. 6, at step 615, computing device 104 produces (or updates) a neonatal profile 116 as a function of neonatal bundle 112. Neonatal profile 116 includes any of the neonatal profile 116 as described above, in reference to FIGS. 1-5. Neonatal profile 116 is produced by obtaining a neonatal functional goal 120. Neonatal functional goal 120 includes any of the neonatal functional goal 120 as described above, in reference to FIGS. 1-5. Neonatal profile 116 is produced by receiving a neonatal recommendation 124. Neonatal recommendation 124 includes any of the neonatal recommendation 124 as described above, in reference to FIGS. 1-5. Neonatal recommendation 124 is received as a function of a neonatal database 128. Neonatal database 128 includes any of the neonatal database 128 as described above, in reference to FIGS. 1-5. Neonatal profile 116 is produced as a function of neonatal functional goal 120 and neonatal recommendation 124 using a neonatal machine-learning model 132. Neonatal machine-learning model 132 includes any of the neonatal machine-learning model 132 as described above, in reference to FIGS. 1-5.

Still referring to FIG. 6, at step 620, computing device 104 determines an aliment 136 as a function of neonatal profile 116. Aliment 136 includes any of the aliment 136 as described above, in reference to FIGS. 1-5.

Still referring to FIG. 6, at step 625, computing device 104 generates a nourishment program 140 as a function of aliment 136. Nourishment program 140 includes any of the nourishment program 140 as described above, in reference to FIGS. 1-5.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

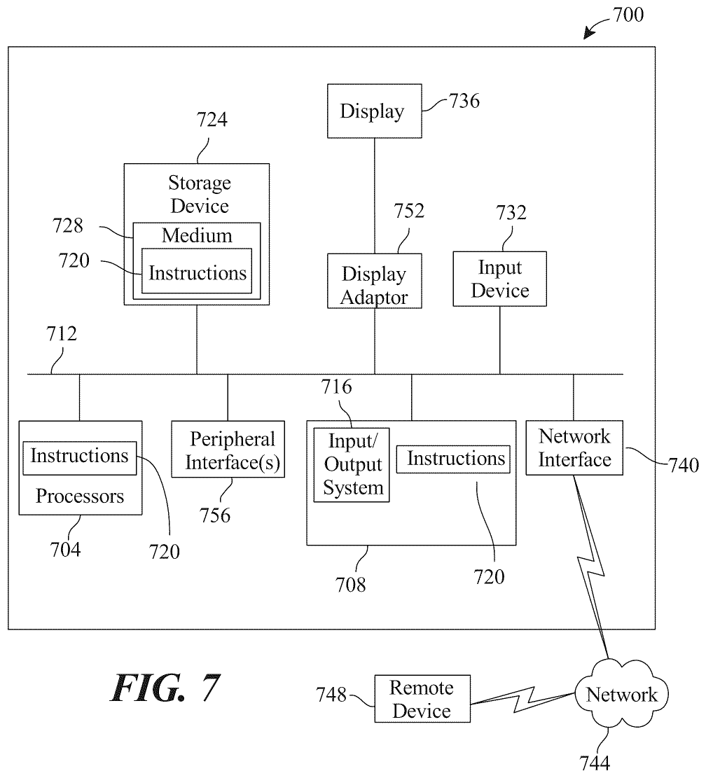

FIG. 7 shows a diagrammatic representation of one embodiment of a computing device in the exemplary form of a computer system 700 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 700 includes a processor 704 and a memory 708 that communicate with each other, and with other components, via a bus 712. Bus 712 may include any of several types of bus structures including, but not limited to, a memory bus, a memory controller, a peripheral bus, a local bus, and any combinations thereof, using any of a variety of bus architectures.

Processor 704 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 704 may be organized according to Von Neumann and/or Harvard architecture as a non-limiting example. Processor 704 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), and/or system on a chip (SoC)

Memory 708 may include various components (e.g., machine-readable media) including, but not limited to, a random-access memory component, a read only component, and any combinations thereof. In one example, a basic input/output system 716 (BIOS), including basic routines that help to transfer information between elements within computer system 700, such as during start-up, may be stored in memory 708. Memory 708 may also include (e.g., stored on one or more machine-readable media) instructions (e.g., software) 720 embodying any one or more of the aspects and/or methodologies of the present disclosure. In another example, memory 708 may further include any number of program modules including, but not limited to, an operating system, one or more application programs, other program modules, program data, and any combinations thereof.

Computer system 700 may also include a storage device 724. Examples of a storage device (e.g., storage device 724) include, but are not limited to, a hard disk drive, a magnetic disk drive, an optical disc drive in combination with an optical medium, a solid-state memory device, and any combinations thereof. Storage device 724 may be connected to bus 712 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 724 (or one or more components thereof) may be removably interfaced with computer system 700 (e.g., via an external port connector (not shown)). Particularly, storage device 724 and an associated machine-readable medium 728 may provide nonvolatile and/or volatile storage of machine-readable instructions, data structures, program modules, and/or other data for computer system 700. In one example, software 720 may reside, completely or partially, within machine-readable medium 728. In another example, software 720 may reside, completely or partially, within processor 704.

Computer system 700 may also include an input device 732. In one example, a user of computer system 700 may enter commands and/or other information into computer system 700 via input device 732. Examples of an input device 732 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 732 may be interfaced to bus 712 via any of a variety of interfaces (not shown) including, but not limited to, a serial interface, a parallel interface, a game port, a USB interface, a FIREWIRE interface, a direct interface to bus 712, and any combinations thereof. Input device 732 may include a touch screen interface that may be a part of or separate from display 736, discussed further below. Input device 732 may be utilized as a user selection device for selecting one or more graphical representations in a graphical interface as described above.

A user may also input commands and/or other information to computer system 700 via storage device 724 (e.g., a removable disk drive, a flash drive, etc.) and/or network interface device 740. A network interface device, such as network interface device 740, may be utilized for connecting computer system 700 to one or more of a variety of networks, such as network 744, and one or more remote devices 748 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 744, may employ a wired and/or a wireless mode of communication. In general, any network topology may be used. Information (e.g., data, software 720, etc.) may be communicated to and/or from computer system 700 via network interface device 740.

Computer system 700 may further include a video display adapter 752 for communicating a displayable image to a display device, such as display device 736. Examples of a display device include, but are not limited to, a liquid crystal display (LCD), a cathode ray tube (CRT), a plasma display, a light emitting diode (LED) display, and any combinations thereof. Display adapter 752 and display device 736 may be utilized in combination with processor 704 to provide graphical representations of aspects of the present disclosure. In addition to a display device, computer system 700 may include one or more other peripheral output devices including, but not limited to, an audio speaker, a printer, and any combinations thereof. Such peripheral output devices may be connected to bus 712 via a peripheral interface 756. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve systems and methods according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions, and additions may be made to that which is specifically disclosed herein without departing from the spirit and scope of the present invention.

What is claimed is:

1. A system for generating a neonatal disorder nourishment program, the system comprising:
a computing device, the computing device configured to:
receive a plurality of infant measurements;
obtain a plurality of neonatal indicator elements by determining a plurality of patterns based on the infant measurements;
identify a neonatal bundle as a function of the plurality of neonatal indicator elements;
update a neonatal profile of an infant as a function of the neonatal bundle, wherein updating the neonatal profile further comprises:
receiving neonatal training data correlating a plurality of neonatal functional goals and a plurality of neonatal recommendations to the neonatal bundle and the neonatal profiles;
training a neonatal machine learning model using the neonatal training data;
inputting the plurality of neonatal functional goals and the plurality of neonatal recommendations to the trained neonatal machine learning model; and
outputting the updated neonatal profile from the trained neonatal machine learning model;
determine an aliment as a function of the updated neonatal profile; and
generate a nourishment program as a function of the aliment.

2. The system of claim 1, wherein an infant measurement comprises an infant sensor measurement comprising an oxygen saturation level of the infant.

3. The system of claim 1, wherein obtaining the plurality of neonatal indicator elements comprises determining a neonatal indicator element comprising a sleep pattern of an infant using a baby monitor comprising a camera and sleep tracking functionality, wherein the baby monitor is configured to provide visual and quantitative insights into a baby's sleep patterns.

4. The system of claim 1, wherein updating the neonatal profile includes determining a neonatal disorder and updating the neonatal profile as a function of the neonatal disorder.

5. The system of claim 4, wherein determining the neonatal disorder comprises:
receiving the neonatal bundle;
training a disorder machine-learning model with a disorder training set correlating at least a neonatal enumeration and an infant organ system effect to a neonatal disorder; and
outputting, using the disorder machine-learning model, the neonatal disorder.

6. The system of claim 4, wherein determining the neonatal disorder comprises:
receiving the neonatal indicator element comprising a photograph of waste;
training an image-based waste machine learning model with training data correlating a plurality of waste related photos to a plurality of neonatal disorders;
outputting, using the image-based waste machine learning model, the neonatal disorder.

7. The system of claim 4, wherein the computing device is further configured to determine a waste remedy as a function of the neonatal disorder.

8. The system of claim 4, wherein the computing device is further configured to classify a user to a cohort of users with similar neonatal disorders.

9. The system of claim 1, wherein determining the aliment further comprises calculating a neonatal phase, wherein the neonatal phase comprises a cognitive phase.

10. The system of claim 1, wherein generating the nourishment program further comprises:
receiving a neonatal outcome; and
generating the nourishment program as a function of the neonatal outcome using a nourishment machine-learning model.

11. A method for generating a neonatal disorder nourishment program, the method comprising:
receiving, using a computing device, a plurality of infant measurements;
obtaining, by the computing device, a plurality of neonatal indicator elements by determining a plurality of patterns based on the infant measurements;
identifying, by the computing device, a neonatal bundle as a function of the plurality of neonatal indicator elements;
updating, by the computing device, a neonatal profile of an infant as a function of the neonatal bundle, wherein updating the neonatal profile further comprises:
receiving neonatal training data correlating a plurality of neonatal functional goals and a plurality of neonatal recommendations to the neonatal bundle and the neonatal profiles;
training a neonatal machine learning model using the neonatal training data;
inputting the plurality of neonatal functional goals and the plurality of neonatal recommendations to the trained neonatal machine learning model; and
outputting the updated neonatal profile from the trained neonatal machine learning model;
determining, by the computing device, an aliment as a function of the updated neonatal profile; and
generating, by the computing device, a nourishment program as a function of the aliment.

12. The method of claim 11, wherein an infant measurement comprises an infant sensor measurement comprising an oxygen saturation level of the infant.

13. The method of claim 11, obtaining the plurality of neonatal indicator elements comprises determining a neonatal indicator element comprising a sleep pattern of an infant using a baby monitor comprising a camera and sleep tracking functionality, wherein the baby monitor is configured to provide visual and quantitative insights into a baby's sleep patterns.

14. The method of claim 11, wherein updating the neonatal profile includes determining a neonatal disorder and updating the neonatal profile as a function of the neonatal disorder.

15. The method of claim 14, wherein determining the neonatal disorder comprises:
receiving the neonatal bundle;
training a disorder machine-learning model with a disorder training set correlating at least a neonatal enumeration and an infant organ method effect to a neonatal disorder; and
outputting, using the disorder machine-learning model, the neonatal disorder.

16. The method of claim 14, wherein determining the neonatal disorder comprises:
receiving the neonatal indicator element comprising a photograph of waste;
training an image-based waste machine learning model with training data correlating a plurality of waste related photos to a plurality of neonatal disorders;
outputting, using the image-based waste machine learning model, the neonatal disorder.

17. The method of claim 14, wherein determining the neonatal disorder further comprises determining a waste remedy as a function of the neonatal disorder.

18. The method of claim 14, wherein determining the neonatal disorder further comprises classifying a user to a cohort of users with similar neonatal disorders.

19. The method of claim 11, wherein determining the aliment further comprises calculating a neonatal phase, wherein the neonatal phase comprises a cognitive phase.

20. The method of claim 11, wherein generating the nourishment program further comprises:
receiving a neonatal outcome; and
generating the nourishment program as a function of the neonatal outcome using a nourishment machine-learning model.
